(19) United States
(12) Patent Application Publication
(10) Pub. No.: US 2003/0157554 A1
(43) Pub. Date: Aug. 21, 2003

(54) Protein-protein complexes and methods of using same

(76) Inventors: Loic Giot, Madison, CT (US); Andrew Eisen, Rockville, MD (US); David A. Lewin, New Haven, CT (US)

Correspondence Address: Ivor R. Elrifi, Esq., Mintz, Levin, Cohn, Ferris,, Glovsky and Popeo, P.C., One Financial Center, Boston, MA, 02111, US

(21) Appl. No.: 10/004,083

(22) Filed: Oct. 30, 2001

Related U.S. Application Data
(60) Provisional application No. 60/244,236, filed on Oct. 30, 2000.

(51) Int. Cl. G01N 33/53; C12Q 1/37; C12N 9/64

(52) U.S. Cl. 435/7.1; 435/226; 435/23

(57) ABSTRACT

The invention provides complexes of at least two polypeptides, and methods of using the same. Purified complexes of two polypeptides are provided, including chimeric complexes, and chimeric polypeptides and complexes thereof are also provided, as are nucleic acids encoding chimeric polypeptides and vectors and cells containing the same. Also provided are methods of identifying agents that disrupt polypeptide complexes, methods of identifying complex or polypeptide in a sample, and for removing the same, methods of determining altered expression of a polypeptide in a subject, and methods of treating/preventing disorders involving altered levels of complex or polypeptide.

### RELATED APPLICATIONS

This application claims priority from U.S. S No. 60/244,236, filed Oct. 30, 2000. The contents of this application is incorporated by reference in its entirety.

### FIELD OF THE INVENTION

The invention relates generally to polypeptides and to complexes of two or more polypeptides, as well as to methods of use thereof.

### BACKGROUND OF THE INVENTION

Most, if not all, biologically important activities are mediated at the tissue, cellular, and subcellular level, at least in part, by interactions between one or more proteins. These biologically important activities can include, e.g., anabolic activities and catabolic activities. Interacting proteins or polypeptides can form a complex. Failure to form a given polypeptide complex can result in deleterious consequences to a cell or individual. Conversely, the inappropriate formation of a given polypeptide complex can likewise be undesirable.

The identification of protein complexes associated with specific biological activities can be used to identify or prevent conditions associated with the absence or presence of these complexes.

### SUMMARY OF THE INVENTION

The invention is based, in part, upon the identification of protein-protein interactions in the yeast S. cerevisiae and humans. Interacting proteins present in complexes according to the invention are shown in, e.g., Table 1.

In one aspect, the invention provides a purified complex including a first polypeptide encoded by the nucleotide sequence recited in Table 1, column 2, and a second polypeptide that includes the corresponding polypeptide encoded by the nucleotide sequence recited in Table 1, column 3.

The invention also provides purified complexes of a first and a second polypeptide. The first polypeptide is a polypeptide functionally classified through GeneCalling™ (as described in U.S. Pat. No. 5,871,697, which is incorporated hereby reference) as an obesity, Type-II diabetes, or hypertension-related protein. The second polypeptide is the corresponding polypeptide encoded by the nucleotide sequence recited in Table 1, column 3.

The invention also provides a purified complex of a first and second polypeptide, where at least one of the polypeptides is an insulin-signaling, vesicular-trafficking, calcium-binding, or glycogen-binding protein.

In a further aspect, the invention provides chimeric polypeptides having six or more amino acids of a first polypeptide covalently linked to six or more amino acids of a second polypeptide. In some embodiments, the chimeric polypeptides are yeast-yeast chimeras, while in others the chimeric polypeptides are human-human or yeast-human chimera. In some embodiments, the first polypeptide is selected from the polypeptides recited in Table 1, column 2 and the second polypeptide is selected from the polypeptides recited in Table 1, column 3. Nucleic acids encoding chimeric polypeptides, and vectors and cells containing the same, are also provided.

In yet another aspect, the invention provides an antibody which specifically binds polypeptide complexes according to the invention. The antibody preferably binds to a complex comprising one or more polypeptides with greater affinity than its affinity for either polypeptide that is not present in the complex.

Also provided by the invention are kits containing in one or more containers, reagent which can specifically detect the complexes of the invention. In one embodiment, the reagent is a complex-specific antibody, while in other embodiments the reagent is an antibody specific for the first or second polypeptides of the complex.

In another aspect, the invention provides pharmaceutical compositions including the complexes described herein. Such compositions are formulated to be suitable for therapeutic administration in the treatment of deficiencies or diseases involving altered levels of the complexes of the invention.

In still another aspect, the invention provides methods of identifying an agent which disrupts a polypeptide complex by providing a complex described herein, contacting the complex with a test agent, and detecting the presence of a polypeptide displaced from the complex. In certain embodiments, the complex includes at least one polypeptide comprising a microtubule or microtubule-associated protein, a heme biosynthesis protein, or a cell wall or cell-wall synthesis protein.

In a further aspect, the invention provides a method for inhibiting the interaction of a protein with a ligand by contacting a complex of the protein and ligand with an agent that disrupts the complex. In certain embodiments, the protein is a vesicle trafficking associated protein, a phosphatase I protein, or a calcium binding protein, and the ligand is a corresponding interacting polypeptide described herein.

In yet another aspect, the invention provides a method of identifying a polypeptide complex in a subject by providing a biological sample from the subject and detecting, if present, the level of a complex, described herein, in the subject.

Also provided by the invention is a method for detecting a polypeptide in a biological sample by providing a biological sample containing a first polypeptide, and contacting the sample with a second polypeptide under conditions suitable to form a polypeptide complex.

In another aspect, the invention provides a method for removing a first polypeptide from a biological sample by providing a biological sample including the first polypeptide, contacting the sample with a second polypeptide under conditions suitable for formation of a polypeptide complex, and removing the complex, thereby effectively removing the first polypeptide. In certain embodiments, the first polypeptide is selected from, or includes, the polypeptides recited in Table 1, column 2 and the second polypeptide is selected from, or includes, the polypeptides recited in Table 1, column 3.

In a further aspect, the invention provides a method for determining altered expression of a polypeptide in a subject by providing a biological sample from the subject, measuring the level of polypeptide complex in the sample, and comparing the level of the complex in the sample to the level of complex in a reference sample with a known polypeptide expression level.

In a still further aspect, the invention provides a method of treating or preventing a disease or disorder involving altered levels of a complex described herein or a polypeptide described herein, by administering, to a subject in need thereof, a therapeutically-effective amount of at least one molecule that modulates the function of the complex or polypeptide. In one embodiment, the agent modulates the function of a polypeptide selected from the polypeptides recited in Table 1.

In one aspect, the invention provides an isolated nucleic acid molecule that includes the sequence of SEQ ID NO: 1, 2, 3, 4, 5, 6, 7, 8, 9, 11, 12, or 13 or a fragment, homolog, analog or derivative thereof. The nucleic acid can include, e.g., a nucleic acid sequence encoding a polypeptide at least 85% identical to a polypeptide encoded by SEQ ID NO: 1, 2, 3, 4, 5, 6, 7, 8, 9, 11, 12, or 13. The nucleic acid can be, e.g., a genomic DNA fragment, or a cDNA molecule.

Also included in the invention is a vector containing one or more of the nucleic acids described herein, and a cell containing the vectors or nucleic acids described herein.

The invention is also directed to host cells transformed with a vector comprising any of the nucleic acid molecules described above.

In another aspect, the invention includes a pharmaceutical composition that includes a NOVX nucleic acid and a pharmaceutically acceptable carrier or diluent.

In a further aspect, the invention includes a substantially purified NOVX polypeptide, e.g., any of the NOVX polypeptides encoded by an NOVX nucleic acid, and fragments, homologs, analogs, and derivatives thereof. The invention also includes a pharmaceutical composition that includes an NOVX polypeptide and a pharmaceutically acceptable carrier or diluent.

In still a further aspect, the invention provides an antibody that binds specifically to an NOVX polypeptide. The antibody can be, e.g., a monoclonal or polyclonal antibody, and fragments, homologs, analogs, and derivatives thereof. The invention also includes a pharmaceutical composition including NOVX antibody and a pharmaceutically acceptable carrier or diluent. The invention is also directed to isolated antibodies that bind to an epitope on a polypeptide encoded by any of the nucleic acid molecules described above.

The invention also includes kits comprising any of the pharmaceutical compositions described above.

In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. All patents and publications cited in this specification are incorporated by reference herein in their entirety.

### DETAILED DESCRIPTION OF THE INVENTION

The invention provides complexes of interacting polypeptides which have not heretofore been shown to interact directly, as well as methods of using these complexes.

Five genes, NAPA1 (Genbank ID: U39412), VAMP2 (Genbank ID: M36201), PPP1CC (Genbank ID: L07395), S100A1 (Genbank ID: M64210) and S100B (Genbank ID: J05600) (hereinafter referred to as “baits”), were identified in GeneCalling™ studies as related to type II diabetes and were cloned in a yeast two-hybrid system. By screening human brain, muscle, liver, and kidney cDNA libraries, 31 interacting proteins were found. Eleven of the 31 interacting proteins are novel. Through a systematic 1×1 assay between the 5 baits and the 31 preys, 58 total interactions were found. The multiple partner associations observed between these proteins provide a high degree of reliability of the biological relevance of most of the interactions found. These interacting pairs include (i) interactions that place novel proteins in a biological context, (ii) novel interactions between proteins involved in the same biological function, and (iii) novel interactions that link together biological functions into larger cellular processes.

Some newly disclosed interactions place functionally unclassified proteins in a biological context. For example, 11 novel proteins, (NOV 11-1, which are collectively referred to as “NOVX”) were observed to interact with at least 1 of the 5 bait proteins which are implicated in type II diabetes.

Also included in the interactions are complexes of two or more proteins involved in functional pathways for which direct interactions have not been described previously. For example, the invention has linked proteins involved in muscle activity to upstream components of the GLUT4 vesicle delivery by showing interaction of the S100 proteins with a new allele Profilin and a vacuolar proton pump. This connection suggests the beneficial effect of muscle activity on glucose uptake observed in type II diabetes.

New insights into novel interactions between proteins involved in the same biological function are also provided. The invention discloses a novel component in vesicle trafficking with the LZIP gene product, which has the same interaction pattern as Syntaxin proteins. In addition, LZIP (Genbank ID: AF029674), with S100A1 (Genbank ID: M65210) and S100B (Genbank ID:J05600) interacts with a detoxification enzyme, NQO2 (Genbank ID: J02888), which protects cells against oxidative stress.

The complexes disclosed herein are useful, inter alia, in identifying agents which modulate cellular processes in which one or more members of the complex have previously been associated. For example, VAMP2-SNAP25A (Protein Pair: 41) as shown in Table 1, have both been implicated in the transport of vesicles containing the glucose transporter GLUT4 from the cytoplasm to the membrane. Accordingly, new agents which modulate vesicle trafficking can be identified by evaluating the ability of a test agent to affect formation or dissolution of a complex of Protein Pair 41.

Complexes according to the invention can also be used in methods for identifying a desired polypeptides in a biological sample by forming a complex of a first polypeptide and a second polypeptide that interacts with the first polypeptide. The presence of the complex indicates that the sample contains the first polypeptide.

These utilities, as well as additional utilities, are discussed in greater detail below.

Novel Nucleic Acids

Below follows additional discussion of the eleven novel nucleic acid sequences shown to interact with diabetes-related bait proteins. The sequences listed below represent the longest contigs resulting from the assembly of all interactors identifying the same cDNA. Underlined sequences represent coding regions and sequences in bold font represent overlapping sequences. Start codons are both italicized and underlined, and the termination codons are both italicized and bolded.

| | |
|---|---|
| NOV1 (Novel_C_30816070) | |
| NOV1 is a novel 374 bp gene fragment. | |
| The nucleic acid has the following sequence: | |
| TTTAGTAGACCTCGTAAACTTTATAAACATTCAAGTACTTCCTCGCGTATTGCTAAAGGA | (SEQ ID NO:1) |
| GGAGTTGACCACACCAAAATGAGTCTACATGGTGCTAGTGGGGGACATGAGAGATCAAGA | |
| GATAGACGAAGGTCAAGTGACAGATCACGAGATTCATCTCATGAAAGAACGGAGTCTCAG | |
| CTCACTCCTTGTATTAGAAATGTGACTTCTCCAACACGACAGCACCATGTTGAACGAGAA | |
| AAAGATCACAGTTCCTCTCGTCCAAGCAGTCCGCGTCCTCAAAAAGCATCCCCAAATGGT | |
| TCCATTAGCAGTGCTGGGAACAGCAGCAGAAACAGTAGTCAGTCAAGTTCAGATGGTAGC | |
| TGTAAGACAGCTGG. | |
| Below represents the extended version of NOV1, | |
| where the extension is italicized: | |
| NGGNGCTCTGGCCCCGGCCTTTGCCCCAATCTTGTGTGGGCACTGAAGGGGGACTACAGG | (SEQ ID NO:2) |
| TTCGAGAGTTATGGGTGCTACATGTGTGCTTTCAGAGCAGTAGTGTGAGGAAGCTTGGAG | |
| TGGG [symbol] GCAGGACGGCCTCATCCCTATGATGGTAACTCCAGTGATCCAGAGAATTGGGA | |
| TCGGAAATTGCATAGTAGACCTCGTAAACTTTATAAACATTCAAGTACTTCCTCGCGTAT | |
| TGCTAAAGGAGGAGTTGACCACACCAAAATGAGTCTACATGATGCTAGTGGGGGACATGA | |
| GAGATCAAGAGATAGACGAAGGTCAAGTGACAGATCACGAGATTCATCTCATGAAAGAAC | |
| GGAGTCTCAGCTCACTCCTTGTATTAGAAATGTGACTTCTCCAACACGACAGCACCATGT | |
| TGAACGAGAAAAAGATCACAGTTCCTCTCGTCCAAGCAGTCCGCGTCCTCAAAAAGCATC | |
| CCCAAATGGTTCCATTAGCAGTGCTGGGAACAGCAGCAGAAACAGTAGTCAGTCAAGTTC | |
| AGATGGTAGCTGTAAGACAGCTGG. | |
| NOV2 (Novel_B_49951007) | |
| NOV2 is a novel 486 bp gene fragment. | |
| The nucleic acid has the following sequence: | |
| CGAGTACAGATACAACTGGATGGCTCCTTCCTTGCGCCAAGAGAGGTTTGCCTTTAAGAT | (SEQ ID NO:3) |
| CTCACCAAAGCCCAGCAAACCACTGAGGCCTTGTATTCAGCTGAGCAGCAAGAATGAAGC | |
| CAGTGGAATGGTGGCCCCGGCTGTCCAGGAGAAGAAGGTGAAAAAGCGGGTGTCCTTCGC | |
| AGACAACCAGGGGCTGGCCCTGACAATGGTCAAAGTGTTCTCGGAATTCGATGACCCGCT | |
| AGATATGCCATTCAACATCACCGAGCTCCTAGACAACATTGTGAGCTTGACGACAGCAGA | |
| GAGCGAGAGCTTTGTTCTGGATTTTTCCCAGCCCTCTGCAGATTACTTAGACTTTAGAAA | |
| TCGACTTCAGGCCGACCACGTCTGCCTTGAGAACTGTGTGCTCAAGGACAAGGCCATGCA | |
| GGCACTG [symbol] AGGTTCAGAACCTCGCATTTGAGAAGACCGTGAAAATAGGATGACGTCGA | |
| CACCTG. | |
| NOV3 (Novel_IP5) | |
| NOV3 is a novel 376 bp gene fragment. | |
| The nucleic acid has the following sequence: | |
| GCATCAAAATTAAGAAGAAAAAAAAAGTACTGTCACCTACGGCTGCCAAGCCAAGCCCCT | (SEQ ID NO:4) |
| TTGAAGGGAAAACGAGCACAGAACCAAGCACAGCCAAACCTTCTTCCCCAGAACCAGCAC | |
| CACCTTCTGAGGCAATGGACGCAGACCGTCCAGGCACCCCGGTTCCCCCTGTTGAAGTCC | |
| CGGAGCTCATGGATACAGCCTCTTTGGAGCCAGGAGCTCTGGATGCCAAGCCAGTGGAGA | |
| GTCCTGGAGATCCTAACCAACTGACCCGGAAAGGCAGGAAGAGGAAAAGTGTGACATGGC | |
| CTGAGGAAGGCAAACTGAGAGAATATTTCTATTTTGAATTGGATGAAACTGAACGAGTAA | |
| ATGTGAATAAGATCAA. | |
| NOV4 (Novel_D_47738563) | |
| NOV4 is a novel 479 bp gene fragment. | |
| The nucleic acid has the following sequence: | |
| CAGAGGCAGGTTTGCTACACAGGAGCGACGACGCAGGCGGCGGCCCCAGCGACTCGCAAC | (SEQ ID NO:5) |
| TGCCTCCCTGACCACAGCGGCCACCGCCCAACACCCCCGAGAAGCCATCGCCACCACCGG | |
| CAGGAGAACCTAGGGTCCATAAAGCCATCTTCGCGATCGACTAAAGCTACGTCAACAACT | |
| ATGGCGGGCGACGGGCGGCGGGCAGAGGCGGTGCGGGAAGGATGGGGTGTGTACGTCACC | |
| CCCAGGGCCCCCATCCGAGAGGGAAGGGGCCGGCTCGCCCCTCAAAATGGCGGCAGCAGC | |
| GATGCGCCTGCGTACAGAACTCCTCCGTCGCGCCAGGGCCGGCGGGAAGTGAGGTTCTCG | |
| GACGAGCCGCCAGAAGTGTACGGCGACTTCGAGCCCCTGGTGGCCAAAGAAAGGTCCCCG | |
| GTGGGAAAACCAACCCGGCTACAAGAGTCCGGCTCGATTCTGCGAAAGAGAAGTAGAGA. | |
| NOV5 (Novel_66002935) | |
| NOV5 is a novel 474 bp gene fragment. | |
| The nucleic acid has the following sequence: | |
| GGCGACTCCGGGGAGGCCGGACACGTCTTTGATGATTTCTCAAGCGACGCCGTTTTCATC | (SEQ ID NO:6) |
| CAGCTCGATGACATGAGCTCGCCACCTTCTCCCGAAAGCACAGACTCTTCCCCGGAGCGA | |
| GACTTCCCACTGAAGCCTGCGTTGCCCCCAGCCAGCCTGGCCGTGGCCGCCATCCAGAGG | |
| GAGGTGTCATTGATGCACGATGAAGACCCTTCGCAGCCCCCACCCCTGCCAGAGGGCACC | |
| CAGGAGCCACATTTGCTCAGGCCGGACGCGGCTGAGAAGGCTGAGGCACCCAGTTCCCCG | |
| GATGTGGCGCCTGCGGNGAAGGAAGACAGCCCCTCTGCGAGTGGGAGGGTACAGGAGGCA | |
| GCCCGGCCTGAGGAGGTGGTTTCGCAGACCCCCCTGCTGCGGTCCAGAGCCCTGGTGAGG | |
| CGGGTCACCTGTAACCTGCAGGAGTCTGAGAGCACGGCCCCGGCGACGACAGAG. | |
| NOV6 (Novel_D_47738671) | |
| NOV6 is a novel 404 bp gene fragment. | |
| The nucleic acid has the following sequence: | |
| TTTTGTCTTTGTATAATAGATGTGATATTTAAAGTCACTGGAAATAGGACAAGTTAATGGA | (SEQ ID NO:7) |
| TGTTTTTATATTTTAATAGAATCATTTATTTCTATGTGTTATGAAATTCACTTAATGATAA | |
| ATTTTTCAACATACTTGCCATTAGAAAACAAAGTATTGCTAAGTACTATAACATATTGGCC | |
| ACTAAAATTCATATTGAGATTATCTTGGTTTCTTGGAAGAGATAGGAATGAGTTCTTATCT | |
| AGTGTTGCAGGCCAGCAAATACAGAGGTGGTTTAATCAAACAGCTCTAGTATGAAGCAAGA | |
| GTAAAGACTAAGGTTTCGAGAGCATTCCTACTCACATAAGTGAAGAAATCTGTCAGATAGG | |
| AATCTAAATATTTATAGTGAGATTGTGAAAGCAACCTT. | |
| NOV7 (Novel_AL031681) | |
| NOV7 is a novel gene fragment. | |
| The nucleic acid has the following sequence: | |
| GAAAAAGGCCTTGTTTTTCAGAAATTCCTGGGTTTCCTGTTAAAAAATCTTAAAGCCCAA | (SEQ ID NO:8) |
| CCT [symbol] GGAATATAGTGCCCCAAAAGGCGGATGCTTCTTCCATTATCTTATTTTCTTTGAT | |
| ACTTTATTTAATTAGATGTTTATAAAGAAATGGGTTTATTTTTCCAGCATAAACCTCAGA | |
| ATTTAAGGAAAGAAAATGATGTCTGTTGTTATAGTTCATTGTTTTGCCTACTCAGCAGAA | |
| GTGATGACTCTTAAAAATTGGCTTTGACCAAAGTTCTCTTGTTTTCAGGGAAAGAACATA | |
| AAAGCTTTTTGAACTACAGCCTTTTTAAAAGAGGGATGGGAGGATATTACAGTAAGAAAT | |
| TAGGCTTTCTAAAAGTATGAAACATCCTTCAACTGGGCTCTCTTGTTAATAGGACATCAT | |
| ATGGTAATAGACTGGTTTGACTAT. | |
| NOV8 (Novel_IP6) | |
| NOV8 is a novel 321 bp gene fragment. | |
| The nucleic acid has the following sequence: | |
| GGCAGCGCTCAGGAAAGGGTTTTTCTCCTCGCGAAGGAAAGAGAGCCGTTGACCATGGTT | (SEQ ID NO:9) |
| GCAACTGGCAGTTTGAGCAGCAAGAACCCGGCCAGCATTTCAGAATTGCTGGACTGTGGC | |
| TATCACCCAGAGAGCCTGCTAAGTGATTTTGACTACTGGGATTATGTTGTTCCTGAACCC | |
| AACCTCAACGAGGTAATATTTGAGGAATCAACTTGGCAGAATTTGGTTAAAATGCTGGAG | |
| AACTGTCTGTCCAAATCAAAGCAAACTAAACTTGGTTGCTCAAAGGTCCTTGTCCCTGAG | |
| AAACTGACGCAGAGAATTGCT. | |
| The open reading frame of NOV8 encodes a 107 | |
| amino acid polypeptide shown below: | |
| GSAQERVFLLAKEREPLTMVATGSLSSKNPASISELLDCG | (SEQ ID NO:10) |
| YHPESLLSDFDYWDYVVPEPNLNEVIFEESTWQNLVKMLE | |
| NCLSKSKQTKLGCSKVLVPEKLTQRIA. | |
| NOV9 (Novel_C_71488908) | |
| NOV9 is a novel 413 bp gene fragment. | |
| The nucleic acid has the following sequence: | |
| GTGGACCAGCTGGAAAAGGAGATTGAGCTGCCCTCGGGCCAGTTGATGGGACTTTTCAAC | (SEQ ID NO:11) |
| CGGATCATCCGCAAAGTTGTGAAGCTATTTAATGAAGTTCAGGAAAAGGCCATTGAGGAG | |
| CAGATGGTGGCAGCGAAGGATGTGGTCATGGAGCCCACGATGAAGACCCTCAGTGACGAC | |
| CTAGATGAAGCAGCAAAGGAATTTCAGGAGAAACACAAGAAGGAAGTAGGGAAGCTGAAG | |
| AGCATGGACCTCTCTGAATACATAATCCGTGGGGACGATGAAGAGTGGAATGAAGTTTTG | |
| AACAAAGCTGGGCCGAACGCCTCGATCATCAGCCTGAAAAGTGACAAGAAAAGGAAGTTA | |
| GAGGCCAAACAAGAAACCCAAACAGAGCAGAAAGTTGAGAAACAGAGAGACAA. | |
| NOV10 (Novel_A_14581444) | |
| NOV10 is a novel 126 bp gene. | |
| The nucleic acid has the following sequence: | |
| TCTCTCTTAAGATTTTTGTGTCTTTTGACTTATATGGAAAGTTATTATACTTGATTGTGA | (SEQ ID NO:12) |
| AATAGGTTTTACTATGATAATTTGCTGACCTACACTTATTTTGTTTTTTTCCTCTAAAAC | |
| AATGTTTTCCTAATGTTTATTTACTTTGCTCTTATGGCTACCCAGTCTGATTCCACATGC | |
| CCTCTTTTGGCCAAACCATCCGCAATTTGTGCTCTCCCTGTCTTCTATCTTTGCCTTCCT | |
| TCTTCTTTCTTAGATATT [symbol] TCCTGGATGCCTCTATTTCTATTCACTGTACTATGGCAT | |
| CAGCTTATAGTCCCTTAATTGCAATGAACTCTATGAAGCTCACATGTCTAGAATATAATC | |
| ACTTTGGCTTCTTTCATGTT. | |
| NOV11 (Novel_IP10) | |
| NOV11 is a novel 472 bp gene fragment. | |
| The nucleic acid has the following sequence: | |
| TTCAAACGCTGCCCGTTCCTAAAGCAAGTCTTGCTTCGGGTCACCTCCCACCTGGTGGCA | (SEQ ID NO:13) |
| GCCAGGGAAAGGGGAAAGGAAGAAGACACTGGAAATGCATGGCCAGCCCCC [symbol] GGGCAT | |
| GAGGAAGGAGCCTTCAGGTGGCCCACAAAGCCCTAGCTCTGGGCCAGGGGCTCTGGGGGG | |
| CTGAGGGGACCAGACTGGGTGCAGGGCCTTGGGAGCTGCCAGCCTCCTTCCCACTGGGCT | |
| TCCGCAGAACTGGGACTCTCACTTCAGGGGCCACCACATCCCTCCTCTCTGCTTCTCCCC | |
| CCAGATCAAAGGGTACCCTCCCACGGTTGGCAGGGCCTGGCTGAGTGCCTCTAGCACCCT | |
| TTGTGCCCACCACAGGCGGTCCCAGGAAGGGCAGCAAGGTCAGACCATTCCTCATTGAAA | |
| ACCGTGGCTAGGGCACAGGGCTCTGATCTGAAGGAGTGACAGATATGTCACA. | |

Purified Polypeptide Complexes

In one aspect, the invention includes a purified complex that includes two or more polypeptides. In one embodiment, the invention provides purified complexes of two or more polypeptides. One of the polypeptides includes a polypeptide selected from the polypeptides recited in Table 1, column 2 and another includes a polypeptide selected from the polypeptides recited in Table 1, column 3. In some embodiments the first and second polypeptides of the complex are the polypeptides enumerated in Table 1. In some embodiments a first polypeptide is listed as a “bait” polypeptide and a second polypeptide is denoted as “prey” polypeptide, while in other embodiments the first polypeptide corresponds to a “prey” polypeptide and the second is a “bait” polypeptide.

By “corresponding polypeptide” is meant, with reference to Table 1, the polypeptide recited in the same row, reading across from left-to-right or right-to-left, as a specific selected peptide. For example, in Table 1, row 1, the corresponding polypeptide of PPP1CC is PPP1CC-NOV1. These protein pairs are designated as Protein Pair ID: 1, as is indicated in Table 1.

Also as used herein, “protein” and “protein complex” are used synonymously with “polypeptide” and “polypeptide complex.” A “purified” polypeptide, protein or biologically active portion thereof is substantially free of cellular material or other contaminating proteins from the cell or tissue source from which the polypeptide is derived, or substantially free from chemical precursors or other chemicals when chemically synthesized. The language “substantially free of cellular material” includes preparations of protein in which the protein is separated from cellular components of the cells from which it is isolated or recombinantly produced. In one embodiment, the language “substantially free of cellular material” includes preparations of polypeptide complex having less than about 30% (by dry weight) of noncomplex proteins (also referred to herein as a “contaminating protein”), more preferably less than about 20% of contaminating protein, still more preferably less than about 10% of contaminating protein, and most preferably less than about 5% non-complex protein. When the polypeptide or complex is recombinantly produced, it is also preferably substantially free of culture medium, i.e., culture medium represents less than about 20%, more preferably less than about 10%, and most preferably less than about 5% of the volume of the protein preparation.

TABLE 1
Protein Interactions Identified

| Protein | | | | Novel | Novel | Self- |
|---|---|---|---|---|---|---|
| Pair ID: | Bait | Prey | Source | Protein | Interaction | Binding |
| 1 | PPP1CC (Genbank | PPP1CC-NOV1 | Screen | Yes | Yes | |
| | ID: L07395) | (Novel_C_30816070) | | | | |
| 2 | PPP1CC(Genbank | NOV2 | Screen | Yes | Yes | |
| | ID: L07395) | (Novel_B_49951007 | | | | |
| | | (hPPP1R4)) | | | | |
| 3 | PPP1CC (Genbank | NOV3 | Screen | Yes | Yes | |
| | ID: L07395) | (Novel_IP5) | | | | |
| 4 | PPP1CC (Genbank | NOV 4 | Screen | Yes | Yes | |
| | ID: L07395) | (Novel_D_47738563 | | | | |
| | | (hLAP1C)) | | | | |
| 5 | PPP1CC (Genbank | NOV5 | Screen | Yes | Yes | |
| | ID: L07395) | (Novel_66002935) | | | | |
| 6 | S100A1 (Genbank ID: | S100-NOV6 (Novel_{—} | Screen | Yes | Yes | |
| | M65210) | D_47738671) | | | | |
| 7 | S100A1 (Genbank ID: | S100-NOV7 | Screen | Yes | Yes | |
| | M65210) | (Novel_AL031681) | | | | |
| 8 | S100A1 (Genbank ID: | S100-NOV8 | Screen | Yes | Yes | |
| | M65210) | (Novel_IP6) | | | | |
| 9 | S100B (Genbank ID: | S100B-NOV9 | Screen | Yes | Yes | |
| | J05600) | (Novel_C_71488908) | | | | |
| 10 | S100B (Genbank ID: | S100B-NOV10 | Screen | Yes | Yes | |
| | J05600) | (Novel_A_14581444) | | | | |
| 11 | S100B (Genbank ID: | S100B-NOV11 | Screen | Yes | Yes | |
| | J05600) | (Novel_IP10) | | | | |
| 12 | NOV2 | NOV2 | 1 × 1 Mating | | Yes | Yes |
| | (Novel_B_49951007 | | | | | |
| | (hPPP1R4)) | (hPPP1R4)) | | | | |
| 13 | 53BP2 (Genbank ID: | NOV4 | 1 × 1 Mating | | Yes | |
| | u58334) | (Novel_D_47738563) | | | | |
| | | (hLAP1C) | | | | |
| 14 | 53BP2 (Genbank ID: | S100B-NOV10 | 1 × 1 Mating | | Yes | |
| | u58334) | (Novel_A_14581444) | | | | |
| 15 | 53BP2 (Genbank ID: | S100B-NOV11 | 1 × 1 Mating | | Yes | |
| | u58334) | (Novel_IP10) | | | | |
| 16 | S100B-NOV9 | S100B-NOV10 | 1 × 1 Mating | | Yes | |
| | (Novel_C_71488908) | (Novel_A_14581444) | | | | |
| 17 | PPP1CC-NOV1 | PPP1CC-NOV1 | 1 × 1 Mating | | Yes | Yes |
| | (Novel_30816070) | (Novel_30816070) | | | | |
| 18 | 53BP2 (Genbank ID: | S100B-NQO2 | 1 × 1 Mating | | Yes | |
| | u58334) | (J02888) | | | | |
| 19 | PPP1CC (Genbank | PPP1R10 (Genbank | Screen | | | |
| | ID: L07395) | ID: Y13247) | | | | |
| 20 | PPP1CC (Genbank | KIAA0305 (Genbank | Screen | | Yes | |
| | ID: L07395) | ID: AB002303) | | | | |
| 21 | PPP1CC (Genbank | STAU (Genbank ID: | Screen | | Yes | |
| | ID: L07395) | AF061939) | | | | |
| 22 | PPP1CC (Genbank | 53BP2 (Genbank ID: | Screen | | | |
| | ID: L07395) | u58334) | | | | |
| 23 | PPP1CC (Genbank | PPP1R5 (Genbank | Screen | | | |
| | ID: L07395) | ID: Y18207) | | | | |
| 24 | PPP1CC (Genbank | PPP1CC (Genbank | 1 × 1 Mating | | | Yes |
| | ID: L07395) | ID: L07395) | | | | |
| 25 | NAPA1 (Genbank ID: | IP2 (Genbank ID: | Screen | | Yes | |
| | U39412) | AC005875) | | | | |
| 26 | NAPA1 (Genbank ID: | LZIP (Genbank | Screen | | Yes | |
| | U39412) | ID: AF029674) | | | | |
| 27 | NAPA1 (Genbank ID: | SNAP29 (Genbank | Screen | | Yes | |
| | U39412) | ID: AF115436) | | | | |
| 28 | NAPA1 (Genbank ID: | SYN16 (GenBank | Screen | | Yes | |
| | U39412) | ID: NM_003763) | | | | |
| 29 | VAMP2 (Genbank | IP2 (Genbank ID: | 1 × 1 Mating | | Yes | |
| | ID: M36201) | AC005875) | | | | |
| 30 | VAMP2 (Genbank | LZIP (Genbank | 1 × 1 Mating | | Yes | |
| | ID: M36201) | ID: AF029674) | | | | |
| 31 | VAMP2 (Genbank | SNAP29 (Genbank | 1 × 1 Mating | | Yes | |
| | ID: M36201) | ID: AF115436) | | | | |
| 32 | VAMP2 (Genbank | SYN16 (GenBank | 1 × 1 Mating | | Yes | |
| | ID: M36201) | ID: NM_003763) | | | | |
| 33 | SNAP29 (Genbank | SYN4 (Genbank ID: | 1 × 1 Mating | | Yes | |
| | ID: AF115436) | u07158) | | | | |
| 34 | SNAP29 (Genbank | LZIP (Genbank | 1 × 1 Mating | | Yes | |
| | ID: AF115436) | ID: AF029674) | | | | |
| 35 | SNAP29 (Genbank | SNAP29 (Genbank | 1 × 1 Mating | | Yes | Yes |
| | ID: AF115436) | ID: AF115436) | | | | |
| 36 | SNAP29 (Genbank | SYN16 (GenBank | 1 × 1 Mating | | Yes | |
| | ID: AF115436) | ID: NM_003763) | | | | |
| 37 | VAMP2 (Genbank | SYN4 (Genbank ID: | 1 × 1 Mating | | Yes | |
| | ID: M36201) | u07158) | | | | |
| 38 | LZIP (Genbank ID: | NQO2 (Genbank ID: | 1 × 1 Mating | | Yes | |
| | AF029674) | J02888) | | | | |
| 39 | NAPA1 (Genbank ID: | SYN4 (Genbank ID: | Screen | | | |
| | U39412) | u07158) | | | | |
| 40 | NAPA1 (Genbank ID: | SNAP25A (Genbank | 1 × 1 Mating | | | |
| | U39412) | ID: D21267) | | | | |
| 41 | VAMP2 (Genbank | SNAP25A (Genbank | Screen | | | |
| | ID: M36201) | ID: D21267) | | | | |
| 42 | SNAP25A | SYN4 (Genbank ID: | 1 × 1 Mating | | | |
| | | u07158) | | | | |
| 43 | S100A1 (Genbank ID: | Fibrinogen | Screen | | Yes | |
| | M65210) | (Genbank ID: | | | | |
| | | M58569) | | | | |
| 44 | S100A1 (Genbank ID: | RanBPM (Genbank | Screen | | Yes | |
| | M65210) | ID: AB008515) | | | | |
| 45 | S100A1 (Genbank ID: | Profilin II-SV | Screen | | Yes | |
| | M65210) | (GenBank ID: | | | | |
| | | A10967919) | | | | |
| 46 | S100B (Genbank ID: | Fibrinogen | Screen | | Yes | |
| | J05600) | (Genbank ID: | | | | |
| | | M58569) | | | | |
| 47 | S100B (Genbank ID: | KIAA0629 (Genbank | Screen | | Yes | |
| | J05600) | ID: AB014529) | | | | |
| 48 | S100B (Genbank ID: | ATPase (ATP6N1) | Screen | | Yes | |
| | J05600) | (Genbank ID: | | | | |
| | | u73006) | | | | |
| 49 | S100B (Genbank ID: | Synphilin (Genbank | Screen | | Yes | |
| | J05600) | ID: AF076929) | | | | |
| 50 | S100B (Genbank ID: | NQO2 (Genbank ID: | Screen | | Yes | |
| | J05600) | J02888) | | | | |
| 51 | S100B (Genbank ID: | FHOS (Genbank ID: | Screen | | Yes | |
| | J05600) | AF113615) | | | | |
| 52 | S100A1 (Genbank ID: | S100B-NOV10 | 1 × 1 Mating | | Yes | |
| | M65210) | (Novel_A_14581444) | | | | |
| 53 | S100A1 (Genbank ID: | NQO2 (Genbank ID: | 1 × 1 Mating | | Yes | |
| | M65210) | J02888) | | | | |
| 54 | S100A1 (Genbank ID: | FHOS (Genbank ID: | 1 × 1 Mating | | Yes | |
| | M65210) | AF113615) | | | | |
| 55 | S100B (Genbank ID: | RanBPM (Genbank | 1 × 1 Mating | | Yes | |
| | J05600) | ID: AB008515) | | | | |
| 56 | S100A1 (Genbank ID: | S100B (Genbank ID: | 1 × 1 Mating | | | |
| | M65210) | J05600) | | | | |
| 57 | S100B (Genbank ID: | S100A9 (Genbank | Screen | | | |
| | J05600) | ID: M26311) | | | | |
| 58 | S100B (Genbank ID: | S100A6 (Genbank | Screen | | | |
| | J05600) | ID: M14300) | | | | |

In certain embodiments, the first polypeptide is labeled. In other embodiments, the second polypeptide is labeled. In still other embodiments, both the first and second polypeptides are labeled. Labeling can be performed using any art-recognized method for labeling polypeptides. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase. Examples of suitable prosthetic group complexes include streptavidin/biotin and avidin/biotin. Examples of suitable fluorescent materials include umbelliferone, fluorescein, fluorescein isothiocyanate, rhodamine, dichlorotriazinylamine fluorescein, dansyl chloride or phycoerythrin. An example of a luminescent material includes luminol. Examples of bioluminescent materials include luciferase, luciferin, and aequorin, and examples of suitable radioactive material include ¹²⁵I, ¹³¹I, ³⁵S, or ³H.

The invention also includes complexes of two or more polypeptides in which at least one of the polypeptides is present as a fragment of a complex-forming polypeptide according to the invention. For example, one or more polypeptides may include an amino acid sequence sufficient to bind to its corresponding polypeptide. A binding domain of a given first polypeptide can by any number of amino acids sufficient to specifically bind to, and complex with, the corresponding second polypeptide under conditions suitable for complex formation. The binding domain can be the minimal number of amino acids required to retain binding affinity, or may be a larger fragment or derivative of the polypeptides listed in Table 1, column 2. Procedures for identifying binding domains can be readily identified by one of ordinary skill in the art and the procedures described herein. For example, nucleic acid sequences containing various portions of a “bait” protein can be tested in a yeast two hybrid screening assay in combination with a nucleic acid encoding the corresponding “prey” protein.

In other embodiments, the complexes are human ortholog complexes, chimeric complexes, or specific complexes implicated in fungal pathways, as discussed in detail below.

Polypeptides forming the complexes according to the invention can be made using techniques known in the art. For example, one or more of the polypeptides in the complex can be chemically synthesized using art-recognized methods for polypeptide synthesis. These methods are common in the art, including synthesis using a peptide synthesizer. See, e.g., Peptide Chemistry, A Practical Textbook, Bodasnsky, Ed. Springer-Verlag, 1988; Merrifield, Science 232: 241-247 (1986); Barany, et al, Intl. J. Peptide Protein Res. 30: 705-739 (1987); Kent, Ann. Rev. Biochem. 57:957-989 (1988), and Kaiser, et al, Science 243: 187-198 (1989).

Alternatively, polypeptides can be made by expressing one or both polypeptides from a nucleic acid and allowing the complex to form from the expressed polypeptides. Any known nucleic acids that express the polypeptides, whether yeast or human (or chimerics of these polypeptides) can be used, as can vectors and cells expressing these polypeptides. Sequences encoding the human polypeptides as referenced in Table 1 are publicly available, e.g. at the Saccharomyces Genome Database (SGD) and GenBank (see, e.g. Hudson et al., Genome Res. 7. 1169-1173 (1997)). If desired, the complexes can then be recovered and isolated.

Recombinant cells expressing the polypeptide, or a fragment or derivative thereof, may be obtained using methods known in the art, and individual gene product or complex may be isolated and analyzed (See, e.g., e.g., as described in Sambrook et al., eds., MOLECULAR CLONING: A LABORATORY MANUAL, 2nd Ed., Cold Spring Harbor Laboratory Press, Cold Spring Harbor, N.Y., 1989; and Ausubel, et al., eds., CURRENT PROTOCOLS IN MOLECULAR BIOLOGY, John Wiley & Sons, New York, N.Y., 1993). This is achieved by assays that are based upon the physical and/or functional properties of the protein or complex. The assays can include, e.g., radioactive labeling of one or more of the polypeptide complex components, followed by analysis by gel electrophoresis, immunoassay, cross-linking to marker-labeled products. Polypeptide complex may be isolated and purified by standard methods known in the art (either from natural sources or recombinant host cells expressing the proteins/protein complex). These methods can include, e.g., column chromatography (e.g., ion exchange, affinity, gel exclusion, reverse-phase, high pressure, fast protein liquid, etc.), differential centrifugation, differential solubility, or similar methods used for the purification of proteins.

Complexes Useful for Identifying Diabetes Related Agents

The invention further provides complexes of polypeptides useful, inter alia, to identify agents and mechanisms that are involved in diabetes.

Diabetes is known to affect approximately eight million people in the United States. Over 90% of diagnosed diabetics suffer from noninsulin-dependent diabetes mellitus (NIDDM), which is also known as type II diabetes. An additional eight million people may have undiagnosed NIDDM. Obesity, advancing age, a family history of NIDDM, a sedentary lifestyle, a history of gestational diabetes, and the presence of co-morbid conditions such as hypertension or hyperlipidemia are risk factors for this disorder.

NIDDM is associated with functional and biochemical abnormalities in the pancreas, liver and peripheral insulin-sensitive tissues such as skeletal muscle and adipose tissue. The abnormalities can include, e.g. relative, but not absolute deficiency of pancreatic insulin secretion, an increased rate of hepatic glucose production and extreme insulin resistance in peripheral tissues such as adipose and skeletal muscle.

One hypothesis for the pathogenesis of NIDDM suggests that the initial event is not pancreatic failure but the development of peripheral tissue insulin resistance. During this “pre-diabetic state” candidate NIDDM patients actually demonstrate hyperinsulinemia, which is an increase level of insulin in the plasma due to an increase in secretion of insulin by the beta cells of the pancreatic islets. The observed pancreatic insulin deficiency that follows is most likely related to pancreatic burnout from maintaining the hyperinsulinemic state.

Persistent, untreated hyperglycemia can result in, e.g., increased risk of urinary tract infections and dehydration related to polyuria. However, often the most important sequellae of diabetes are its long term complications. Following 15-20 years of poorly managed diabetes, patients are at risk for peripheral vascular disease with risk of limb-amputating gangrene, blindness, myocardial infarction and renal failure.

(i) Vesicle Trafficking

According to the SNAREs hypothesis, vesicle docking proteins (vSNARE) specifically bind to targeted membrane bound proteins (tSNARE) to form a recognition complex. When assembled, this complex recruits general elements of the membrane fusion apparatus, namely SNAP proteins (for stabilization) and NSF (N-ethylmaleimide-sensitive factor).

Four proteins SYN4, VAMP2, NAPA1, SNAP25A are responsible, with other proteins, for the transport of vesicles containing the glucose transporter GLUT4 from the cytoplasm to the membrane. VAMP2 is a vSNARE protein localized on GLUT4 vesicles and SYN4 is a tSNARE protein at the cellular membrane. GLUT4 vesicle translocation is induced upon insulin stimulation in human insulin-responsive tissues like muscle or adipose. A second reservoir of GLUT4 vesicles, which responds to muscle activity instead of insulin, also contains the VAMP2 protein, but the other components, as well as the molecular process leading to their translocation, are still unknown.

All of the known interactions between SYN4, VAMP2, NAPA and SNAP25A have been reconstituted in a 1×1 mating assay. In addition, two components (SYN16 and SNAP29) were found to participate in the same complex or substitute their counterpart (SYN16 for SYN4 and SNAP29 for SNA25A) to generate new NSF receptors. The cellular localization of these two proteins on specific membrane compartments and their expression profile would discriminate between the two hypotheses.

TABLE 2
Vesicle Trafficking-Related Interactions Identified

| | |
|---|---|
| NAPA1 (Genbank ID: | Two interactors obtained from liver contain the same fragment which matches |
| U39412) -IP 2(Genbank | the human genomic clone AC005875. The insert contains a putative ORF (52aa) |
| ID: AC005875) | in frame with the Gal4-AD. This putative protein is 82% homologous to the |
| | putative protein express in frame 2 of the assembly 65706057 and 60-68% |
| | homologous to the retrotransposable L1 element. |
| NAPA1(Genbank ID: | Three interactors obtained from liver contain the same fragment (2 kb) which |
| U39412) - SYN16 | matches the cDNA coding for the human Syntaxin 16 protein. The insert starts at |
| (GenBank ID: | codon 174 (frame 2) and covers the C-terminal domain of the protein [174-307]. |
| NM_003763) | |
| NAPA1(Genbank ID: | Six interactors obtained from muscle contain the same fragment (2 kb) which |
| U39412)-SNAP29 | matches the cDNA coding for the human SNAP29 protein. The insert starts at |
| AF115436) | position −69 (frame 1), adding 23 aa at the junction and should cover the entire |
| | coding region of SNAP29 [1-335 aa]. |
| NAPA1(Genbank ID: | Interactors obtained from brain (24 hits) and Muscle (27 hits) contain the same |
| U39412)-SYN4 (Genbank | fragment which matches the cDNA coding for the Human Syntaxin 4. The insert |
| ID: u07158) | starts at codon 158 (frame 2) covers the C-terminal domain of the protein [158-297] |
| | containing one of the two coiled coil domains [199-222], as well as the |
| | transmembrane domain [276-296]. |
| NAPA1(Genbank ID: | LZIP proteins form a large family of transcription regulators (the cellular |
| U39412)-LZIP (Genbank | counterpart of the viral VP 16 transcription factor) defined by the presence of a |
| ID: AF029674) | basic DNA-binding domain and a leucine zipper that recognize the CRE and AP-1 |
| | elements. However, LZIP behaves similarly to SYN4 and SYN16 by |
| | interacting with NAPA, VAMP2, and SNAP29. Comparison of the three protein |
| | sequences did not reveal a conserved domain to explain this pattern. LZIP is also |
| | binding to the Human water channel aquaporine 3 (AQP3). SYN4 as well as |
| | VAMP2 seem to be involved in the aquaporin-2 vesicle targeting to the apical |
| | plasma membrane of rat renal collecting duct cells. These interactions strongly |
| | suggest that LIZP could be involved in the GLUT4 vesicle trafficking, in |
| | addition to other functions. |
| | Interactors obtained from liver (8 hits) and muscle (5 hit) contain the same |
| | fragment (2 kb), which matches the cDNA coding for the human LZIP protein. |
| | The insert starts at position −174 prior to the start codon and should cover the |
| | complete coding sequence [372 aa]. |
| VAMP2 (Genbank ID: | Six interactors obtained from Brain, with the bait VAMP2-2, contain overlapping |
| M36201)-SNAP25A | 1.5 kb fragments which match the cDNA coding for the human SNAP25A |
| (Genbank ID: D21267) | protein. The five fragments identified, start at positions −135, −90, −81, −60 |
| | and −48 from the ATG and should cover the entire coding region of SNAP25A |
| | (206 aa). |

As described above, in certain embodiments of these complexes contain the binding domains, of the polypeptides recited in Table 2, while other embodiments contain conservative variants of these polypeptides, or polypeptides, which contain the polypeptides recited in Table 2.

Confirmation and 1×1 Tests

To identify additional interactions between the baits and the preys, each bait was cloned in fusion with the Gal4-AD domain and each prey was cloned in fusion with the Gal4-BD domain.

To facilitate these DNA transfers, a new pAD plasmid was engineered such that it contains the same recombination sequences than the pBD plasmid to transfer any insert by gap-repair without PCR. The new plasmid was controlled by sequencing and was tested for its capacity to not affect known interactions.

To transfer the preys into the BD plasmid, the inserts from the AD plasmid were amplified and the PCR product was transferred into the BD plasmid by gap-repair. Four clones for each construct were controlled by PCR sizing and two positives were used for the mating experiments.

The 6 vesicle-trafficking interactions, described above, passed confirmation. Moreover, all 6 interacting proteins interacted with both full-length baits, NAPA and VAMP2-1 (Table 3), indicating that the interacting proteins can form multi-subunit complexes. However, only SYN16 and SNAP25A bind the truncated allele VAMP2-2, indicating different requirement for these proteins to bind VAMP2.

When the 6 interacting proteins were tested against each other (Table 3), 5 additional interactions as well as one self interaction for SNAP29 were identified. Only the interaction between SNPA29 and SYN16 was positive in both orientations.

When tested against other interacting proteins identified during this project, one additional interaction was observed (LZIP-NQO2), confirming the high specificity of the interactions describe above.

TABLE 3
Interactions Between The Proteins Involved In Vesicle Trafficking

| AD | BD FUSIONS | | | | | | | | |
|---|---|---|---|---|---|---|---|---|---|
| FUSIONS | NAPA | VAMP2-1 | VAMP2-2 | SYN4 | AC005875 | LZIP | SNAP29 | SYN16 | SNAP25A |
| NAPA1 | ND | ND | ND | ND | ND | ND | ND | ND | ND |
| VAMP2-1 | ND | ND | ND | ND | ND | ND | ND | ND | ND |
| VAMP2-2 | No | No | No | No | No | No | No | No | No |
| SYN4 | Yes | Yes | No | No | No | No | Yes | No | Yes |
| AC005875 | Yes | Yes | No | No | No | No | No | No | No |
| LZIP | Yes | Yes | No | No | No | No | Yes | No | No |
| SNAP29 | Yes | Yes | No | No | No | No | Yes | Yes | No |
| SYN16 | Yes | Yes | Yes | No | No | No | Yes | No | No |
| SNAP25A | Yes | Yes | Yes | No | No | No | No | No | No |

(ii) Phosphatase I Activity

The human phosphatase 1 (PP1) is essential for cell division, participates in the regulation of glycogen metabolism, muscle contractility and protein synthesis. PP1 activity relies on three catalytic subunits (PPP1CA, PPP1CB and PPP1CC) and at least ten regulatory proteins (PPP1R proteins). Three PP1 regulatory proteins were identified, two of which are glycogen binding proteins, the last being related to the nuclear targeting of PP1 during mitosis. The three proteins described below also contain a conserved motif {(R/K)(V/I)XF} responsible for their binding to the PP1 catalytic subunit. Other PPP1CC-interacting proteins also share this motif, thereby providing evidence for the relevance of their interaction with PPP1CC in the two-hybrid assay.

In addition, a systematic search of the PathCalling™ database indicated that the proteins PPP1R10, STAU, and p53BP2 also interact with the catalytic subunit PPP1CA.

TABLE 4
Phosphatase I Complexes Identified
Novel PPP1CC-Interacting Proteins and Interactions

| | |
|---|---|
| PPP1CC (Genbank ID: | One interactor obtained from brain contains a 1.5 kb fragment which matches |
| L07395)- NOV1 | category C assembly 30816070. The insert contains a putative ORF (155 aa) in |
| (Novel_C_30816070) | frame with Gal4-AD. This putative ORF can be extended by the assembly until |
| | a Met codon. The extended ORF, which is serine/arginine rich, does not contain |
| | any signal peptide, hydrophobic domain, or significant homology to other |
| | proteins. |
| PPP1CC(Genbank ID: | NOV2 is a new human protein homolog to the Rat PPP1R4 protein which is |
| L07395)-NOV2 | another glycogen binding subunit. Thirty interactors obtained from liver contain |
| (Novel_B_49951007)(hPPP1R4) | the same fragment which matches the category B assembly 49951007. The insert |
| | contains a putative ORF of 142aa in frame 2 which is 87% homologous to the |
| | Hepatic Glycogen-Binding subunit protein Phosphatase-1 from Rattus |
| | Norvegicus (also named PPP1R4, q63759). |
| PPP1CC (Genbank ID: | Two interactors obtained from Kidney contain the same fragment coding for the |
| L07395)-NOV3 | Human Betaine Homocystein Methyltransferase (u50929). |
| (Novel_IP5) | |
| PPP1CC(Genbank ID: | PPP1CC-IP9 interactors identified a new human protein homologous to the |
| L07395)-NOV4 | lamina-associated polypeptide 1C from Rat. Comparison of the protein sequence |
| (Novel_D_47738563)(hLAP1C) | with other PPP1CC-IP indicates that hLAP1C contains a Phosphatase 1 binding |
| | site (R/K)(V/I)XF. LAP1C protein has been shown to be involved in the partition |
| | of the nuclear envelop during mitosis. These results suggest that the new protein |
| | hLAP1C may participate in the nuclear targeting of PP1 during mitosis. |
| | Four interactors obtained from muscle contain the same 3.5 kb fragment which |
| | matches the category D assembly 47738563. The insert contains a putative ORF |
| | (99aa) starting by a Met codon at position 181 (frame1) (so adding 60aa at the |
| | junction) which is 77% homologous to the Lamina-associated polypeptide 1C |
| | from Rattus Norvegicus LAP1C (q62741). |
| PPP1CC (Genbank ID: | PPP1CC-IP12 is a new human protein homologous to the CTD-binding SR-like |
| L07395)-NOV5 | protein rA9 from rat. This protein is a member of the Serine rich proteins family |
| (Novel_66002935) | shown to physically and functionally link transcription and pre-mRNA |
| | processing. |
| | One interactor obtained from brain contain a 0.5 kb fragment which is 89% |
| | homologous to the assembly 66002935. The insert contains a putative ORF in |
| | frame 1 with a Met codon at position 73. The putative protein is 59% |
| | homologous to the CTD-binding SR-like protein rA9 Rattus Norvegicus |
| | (q63625). |

Novel PPP1CC Interactions

| | |
|---|---|
| PPP1CC (Genbank ID: | PPP1R5 is a glycogen binding subunit which targets PP1 to glycogen particles |
| L07395)-PPP1R5 | and inhibits its phosphorylase phosphatase activity. PPP1R5 also interacts with |
| (Genbank ID: Y18207) | glycogen synthase, suggesting a role in connecting enzymes of glycogen |
| | metabolism with PP1. Interactors obtained from brain (6 hits), Liver (14 hits) |
| | and muscle (45 hits) contain overlapping fragments which match the cDNA |
| | coding for the human PPP1R5 protein. Two inserts start at position −85 and −55 |
| | (frame2) from the ATG and should contain the complete coding sequence of |
| | PPP1R5 (318aa) However, there is a natural stop codon (TAG) 24 bp before the |
| | ATG indicating that PPP1R5 is not expressed as a Gal4-AD fusion from these |
| | inserts. Four additional inserts (all from muscle) start at positions +14, +41, +47, |
| | +55 within the coding sequence. |
| PPP1CC (Genbank ID: | Two interactors obtained from muscle contain the same 4 kb fragment which |
| L07395) -KIAA0305 | matches the cDNA coding for the human KIAA0305 protein. The insert starts at |
| Genbank ID: AB002303 | position −48 from the ATG and should cover the complete coding region [1-1540]. |
| | There is a natural stop codon (TAA) 27 bp before the ATG indicating that |
| | KIAA0305 is not expressed as a Gal4-AD fusion. |
| PPP1CC (Genbank ID: | As shown in Table 5, STAU contains a PP1 binding motif. STAU also binds to |
| L07395)-STAU (Genbank | tubulin, which can relate to the ability of PP1 to dephosphorylate tubulin and |
| ID: AF061939) | other microtubules. This suggests that STAU can interfere with other PP1 |
| | functions in addition to those associated with glucose metabolism. |
| | This interaction is novel. Two interactors obtained from brain contain the same |
| | 3.5 kb fragment which matches the cDNA coding for the Human STAU protein. |
| | The insert starts at codon 6 (frame 2) and covers the entire coding region of the |
| | Staufen protein [6-578]. |
| PPP1CC (Genbank ID: | P53BP2 is known to bind PPP1CC and inhibit the PP1 phosphorylase |
| L07395)-53BP2 (Genbank | phosphatase activity, suggesting that PP1 can regulate the activity of p53 through |
| ID: u58334) | its binding to p53BP2. Interactors obtained from brain (4 hits) and kidney (1 hit) |
| | contain the same 2 kb fragment which matches the cDNA coding for the human |
| | p53BP2 protein. The insert starts at codon 582 (frame 2) and should cover the |
| | entire C-terminal domain of 53BP2 [582-1005]. Although this interaction is |
| | generally known, the interaction identified between 53BP2 with PPP1CC is |
| | novel. Published results indicate that “The region required for interaction with |
| | PP1 was shown to be contained within amino acids 297-431 of p53BP2, which |
| | includes two ankyrin repeats”. There is also evidence that a second domain |
| | [582-1005] can interact with PPP1CC. |

Known PPP1CC Interactions

| | |
|---|---|
| PPP1CC (Genbank ID: | hPPP1R10 is a PP1 nuclear targeting subunit. The rat homolog (PNUTS) can |
| L07395)-PPP1R10 | bind the catalytic subunit PPP1CA in the two-hybrid system as well as |
| (Genbank ID: Y13247) | hPPP1R10 binds to the hPPP1CA. One interactor obtained from kidney contains |
| | a 2 kb fragment which matches the cDNA coding for the human PNUTS (or |
| | PPP1R10) protein. The insert starts at codon 272 (frame 3) and covers the C- |
| | terminal domain of PNUTS [272-940]. |

Complexes containing one or more variants of these polypeptides are within the scope of the present invention, as are polypeptides having amino acid sequences which include the polypeptides encoded by the nucleic acid sequences recited in Table 1.

[image]

Confirmation and 1×1 Tests

To identify additional interactions between the baits and the preys, each bait was cloned in fusion with the Gal4-AD domain and each prey was cloned in fusion with the Gal4-BD domain.

To facilitate these DNA transfers, a new pAD plasmid was engineered that contains the same recombination sequences as the pBD plasmid to transfer any insert by gap-repair without PCR. The new plasmid was controlled by sequencing and was tested for its capacity to not affect known interactions.

To transfer the preys into the BD plasmid, the inserts from the AD plasmid were amplified and the PCR product was transferred into the BD plasmid by gap-repair. Four clones for each construct were controlled by PCR sizing and two positives were used for the mating experiments.

The 9 PPP1CC interactions listed above passed confirmation and 3 of the 9 interactions were also positive in the opposite orientation (53BP2, PPP1CC-IP2 and hPPP1R4). When the 9 interacting proteins were tested against each other as shown in Table 6, one additional interaction (53BP2-hLAP1C) as well as two self interactions (PPP1CC-IP2 and hPPP1R4) were identified. PPP1CC was also shown to form a homodimer, confirming previous results suggesting that the catalytic activity of the native glycogen-bound phosphatase resides in a dimer of 38,000-dalton subunits.

When tested against other interacting proteins identified during this project, 3 additional interactions for p53BP2 with three of the S100B interacting proteins (S100B-NOV10, S100B-NOQ2, and S100B-NOV11) were observed. These results suggest a connection between the Phosphatase 1 activity and the S100 regulations. They also indicate that the interactions identified with PPP1CC are highly specific.

TABLE 6
Interactions Between The Proteins Interacting With PPP1CC

| AD | BD FUSIONS | | | | | | | | | |
|---|---|---|---|---|---|---|---|---|---|---|
| FUSIONS | PPP1CC | 53BP2 | IP2-New | PPP1R5 | hPPP1R4 | PPP1R10 | KIAA0305 | hLAP1C | STAU | IP12-New |
| PPP1CC | Yes | Yes | Yes | No | Yes | ND | ND | No | No | No |
| 53BP2 | Yes | No | No | No | No | ND | ND | No | No | No |
| IP2-New | Yes | No | Yes | No | No | ND | ND | No | No | No |
| PPP1R5 | Yes | No | No | No | No | ND | ND | No | No | No |
| PPP1R4 | Yes | No | No | No | Yes | ND | ND | No | No | No |
| PPP1R10 | Yes | ND | ND | ND | ND | ND | ND | ND | ND | ND |
| KIAA0305 | Yes | ND | ND | ND | ND | ND | ND | ND | ND | ND |
| hLAP1C | Yes | Yes | No | No | No | ND | ND | No | No | No |
| STAU | Yes | No | No | No | No | ND | ND | No | No | No |
| IP12-New | Yes | No | No | No | No | ND | ND | No | No | No |

(iii) Calcium Binding

The S100 proteins used as baits in this invention are Calcium/Zinc binding proteins which can form homo- or hetero-dimers. This family contains more than 19 members which, upon Calcium/Zinc binding, associate with their targets to regulate a variety of intracellular activities such as protein phosphorylation, enzyme activities, cell proliferation (including neoplastic transformation) and differentiation, the dynamics of cytoskeleton constituents, the structural organization of membranes, intracellular Ca2+ homeostasis, inflammation, and protection from oxidative cell damage. In addition, several S100 proteins are clustered on human chromosome 1 q21, a region frequently rearranged in several tumors and conserved during evolution. Both S100A1 and S100B are differentially expressed in GeneCalling™ Diabetes studies.

When the two baits, S100A1 and S100B were tested against each other, the two full length proteins interacted together, and with themselves. The capacity of both proteins to form hetero- and homo-dimers has been published. In addition, any combination involving at least one C-terminal truncation failed to give a positive signal. These results are in agreement with other studies showing a conformational change of this domain is needed for the dimerization upon calcium binding. These confirm that the S100 truncation eliminates both the high affinity calcium binding site [63-75], as well as the dimerization domain [75-93].

Two other members of the S100 protein family have been identified as S100B-interacting proteins within the invention: S100A6 and S100A9. S100A9 is known to form a hetero-dimer with S100A8. This complex can bind unsaturated fatty acids with high affinity and is involved in chronic inflammation. As a unique feature in the S100 protein family, this complex can be secreted and has extracellular association with vascular endothelium adjacent to transmigrating leukocytes. In addition to its interaction with the diabetes-related proteins S100A1 and S100B, both S100A6 and S100A9 are highly expressed in nervous tissues and are associated with neurodegenerative disorders. S100A6 is also differentially expressed between normal uveal melanocytes and malignant melanomas.

Several common targets to S100A1 and S100B are known. The phosphoglucomutase protein, which is involved in the glucose metabolism, is inhibited by S100A1 and activated by S100B. The type III intermediate filament (IF) subunits, desmin, and glial fibrillary acidic protein (GFAP) are other common targets to inhibit microtubule (MT) protein assembly and to promote MT disassembly. The proteins, NQO2, FHOS, NOV9, and fibrinogen, are described below in Table 7.

S100A1 has been shown to interact with cytoskeleton proteins (desmin, GFAP and FHOS). Two other S100A1-interacting proteins, Profilin II and RanBPM, are related to the cytoskeleton organization. They have an important role in the GLUT4 vesicle delivery upon insulin stimulation during glucose uptake. In addition, three additional proteins (fibrinogen, NOV10, and ATP6N 1) were identified as specifically interacting with S100A1. These interactions are further described below in Table 7.

Four prey proteins, Synphilin I, ATP6N17, NOV11, and KIAA0629 have been shown to interact with S100B and are further described below in Table 7.

TABLE 7
Calcium Binding Protein Interactions Identified
Novel S100A1 Interacting Proteins and Interactions

| | |
|---|---|
| S100A1 (Genbank ID: M65210) -NOV6 | Three interactors obtained from liver (1 hit) and muscle (2 hits) |
| (Novel_D_47738671) | contain the same 0.3 kb fragment which matches the category D |
| | assembly 47738671 (which is extended of 188 bp by the clone |
| | S100A1-IP2). The insert does not contains a putative ORF in |
| | frame 1. The longest ORF is in frame 2 [nucleotide 95 to 295 |
| | (67 aa)] and does not share significant homology with other |
| | proteins. |
| S100A1(Genbank ID: M65210) - | Three interactors obtained from muscle contains the same 0.4 |
| NOV7(Novel_AL031681) | kb fragment which matches a human genomic clone (862K6) |
| | on chromosome 20q12-13.13 containing the gene SFRS6 |
| | coding for the arginine/serine-rich splicing factor 6 (SRP55-2). |
| | However, the insert is in the opposite orientation compare to |
| | SRP55 and there is no ORF in any of the three frame. The |
| | following 21 aa are in fusion with the Gal-4 BD domain: |
| | EKGLVFQKFLGFLLKNLKAQP (SEQ ID NO: 14) and do not |
| | display any similarity with known protein domains. |
| S100A1(Genbank ID: M65210) - NOV8 | One interactor obtained from muscle contain a 1.7 kb fragment |
| (Novel_IP6) | which does not matches to any known sequences. The insert |
| | contains a putative ORF of 107aa, in frame 1, with a start |
| | codon at position 55. This putative protein does not display |
| | homology with any known proteins or with proteins translated |
| | from the SeqCalling database. |

Novel S100A1 Interactions

| | |
|---|---|
| S100A1 (Genbank ID: M65210) - Fibrinogen | Fibrinogen is known as a risk factors for atherosclerosis in |
| (Genbank ID: M58569) | patients with type 2 diabetes mellitus and recent results have |
| | shown that hyperglycaemia increase the plasma fibrinogen |
| | concentration. |
| | One interactor obtained from liver contains a 0.6 kb fragment |
| | which matches the cDNA coding for the human fibrinogen |
| | alpha subunit protein. The insert starts at codon 345 (frame 1) |
| | and covers an internal domain of around 200aa [345 to ˜545]. |
| S100A1 (Genbank ID: M65210) -RanBPM | RanBPM is a regulatory protein of the small GTPase Ran and |
| (Genbank ID: AB008515) | acts as an ectopic microtubule nucleation sites, resulting in a |
| | reorganization of the microtubule network. |
| | Three interactors obtained from muscle contains the same 1.7 |
| | kb fragment which matches the cDNA coding for the human |
| | RanBPM protein. The insert starts at position −367 (frame 1) |
| | adding 122 aa at the junction and covers the first 440aa of |
| | RanBPM (501aa). |
| S100A1 (Genbank ID: M65210) -Profilin II | Profilin II is an actin binding protein affecting the structure of |
| SV (Genbank ID: AL096719) | the cytoskeleton by inhibiting the actin polymerization. The |
| | localization of profilin is affected by phosphoinositides which |
| | are part of the insulin signaling pathway. Insulin acts on the |
| | surface morphology and cytoskeleton reorganization of |
| | hepatocytes and adipocytes, through the stimulation of the |
| | phosphoinositides 3 kinase, to stimulate vesicle trafficking. |
| | These results suggest that Profilin II is the target of the insulin |
| | pathway in order to deliver the GLUT4 vesicles at the |
| | membrane. The interaction between Profilin II and S100A2 |
| | connect profilin to muscle activity and suggest another way to |
| | stimulate the GLUT4 vesicle delivery independently to insulin. |
| | This model would fit with the increase of glucose uptake |
| | associated with muscle activity. The protein identified in the |
| | screen is a new allele displaying a modified C-terminal domain |
| | compared to the known Profilin II. |
| | Six interactors obtained from muscle contains a 1.3 kb |
| | fragment which matches a human cDNA (DKFZp566N043) |
| | and is 90% homologous to the cDNA coding for the human |
| | Profilin 2 protein. The protein expressed from the insert is 99% |
| | homologous [98 out of 99aa] to the Bos Taurus Profilin II |
| | (q09430) and 90% homologous [90 out of 99aa] to the Homo |
| | Sapiens Profilin II (p35080). By homology, one insert (in |
| | frame 1) starts at aa 8 and five inserts (in frame 1) start at aa38. |
| | All inserts should cover the rest of the protein (139aa). |

Novel S100B Interacting Proteins and Interactions

| | |
|---|---|
| S100B (Genbank ID: J05600)- NOV9 | S100B-Novel_C_71488908 also binds to a S100B- |
| (Novel_C_71488908) | Novel_A_14581444 protein, which is another novel human |
| | protein. The specificity of these interactions suggests that the |
| | four proteins can form a multi-subunit complex. |
| | Six interactors obtained from liver contain the same 0.7 kb |
| | fragment which matches the category C assembly 71488908. |
| | The insert contains a putative ORF of 117aa, in frame 1, with a |
| | start codon at position 46. This putative protein does not |
| | display specific domains or homology with any known |
| | proteins. |
| S100B (Genbank ID: J05600)-NOV10 | One interactors obtained from liver contain a 1.5 kb fragment |
| (Novel_A_14581444) | which matches the category A assembly 14581444. The insert |
| | contains a putative ORF of 42aa, in frame 1, with a start codon |
| | at position 133. This putative protein does not display specific |
| | domains or homology with any known proteins. |
| S100B(Genbank ID: J05600) -NOV11 | Six interactors obtained from muscle contain the same 0.6 kb |
| (Novel_IP10) | fragment which does not match any known sequence or |
| | SeqCalling assembly. The following 37aa are in fusion with |
| | the Ga14-BD domain: |
| | FKRCPFLKQVLLRVTSHLVAARERGKEEDTGNAWPAP |
| | (SEQ ID NO:15). |

Novel S100B Interactions

| | |
|---|---|
| S100B (Genbank ID: J05600)-Fibrinogen | Fibrinogen is known as a risk factors for atherosclerosis in |
| (Genbank ID: M58569) | patients with type 2 diabetes mellitus and recent results have |
| | shown that hyperglycaemia increase the plasma fibrinogen |
| | concentration. |
| | Three interactors obtained from Liver contain overlapping |
| | fragments (1.0; 0.6; 0.3 kb) which matches the cDNA coding |
| | for the human Fibrinogen subunit alpha protein. The three |
| | inserts can be assembled into a 1.5 kb contig. |
| S100B (Genbank ID: J05600) -KIAA0629 | Two interactors obtained from Liver contain a 0.7 kb fragment |
| (AB014529) | which matches the cDNA coding for the putative human |
| | protein KIAA0629. The insert does not contains significant |
| | ORF as the KIAA0629 cDNA. |
| S100B (Genbank ID: J05600)-ATP6N1 | Vacuolar-type H(+) ATPase (ATP6N1) is a non-catalytic |
| (Genbank ID: u73006) | subunit of the proton pump needed for the acidification of |
| | clathrin-coated vesicles. A recent study has shown that specific |
| | inhibition of the vacuolar proton pump by bafilomycin A1 in |
| | adipocytes resulted in the rapid and dose-dependent |
| | translocation of GLUT4 from the cell interior to the membrane |
| | surface mimicking 50 to 65% of the effects of acute insulin |
| | treatment. |
| | One interactors obtained from Liver contain a 1 kb fragment |
| | which matches the cDNA coding for the human Vacuolar-type |
| | H(+) ATPase protein (ATP6N1). The insert starts at position — |
| | 99 (frame 3) adding 33 aa at the junction and should cover a N- |
| | terminal domain [1 to ˜300] of the Vacuolar-type H(+) ATPase |
| | protein (881aa). |
| S100B (Genbank ID: J05600)-Synphilin I | In addition to its interaction with the diabetes-related protein, |
| (Genbank ID: AF076929) | S100B, Synphilin I has been recently identified as a Synuclein |
| | interacting protein by the two-hybrid assay. Synucleins proteins |
| | are implicated in the pathogenesis of Parkinson disease and |
| | may serve to integrate presynaptic signaling and membrane |
| | trafficking. |
| | Four interactors obtained from Kidney contain the same 1 kb |
| | fragment which matches the cDNA coding for the human |
| | Synphilin 1 protein. The insert starts at position 335 (frame 2) |
| | and should cover an internal domain [335 to ˜660] of the |
| | Synphilin 1 protein (919aa). |
| S100B (Genbank ID: J05600)-NQO2 | The Quinone Oxidoreductase protein NQO2 provides |
| (Genbank ID: J02888) | protection to cells against oxidative stress which is one of the |
| | central features of diabetic complications caused by |
| | hyperglycaemia. The NQO2 gene localized to the human |
| | chromosome 6 and displays intensive polymorphism. |
| | Five interactors obtained from Muscle contain the same 0.7 kb |
| | fragment which matches the cDNA coding for the human |
| | Quinone Oxidoreductase 2 protein. The insert starts at position |
| | −214 (frame 1) and should cover a N-terminal domain [1 to |
| | ˜160] of the NQO2 protein (231aa). |
| S100B (Genbank ID: J05600)-FHOS | The FHOS protein has been recently identified as a member of |
| (Genbank ID: AF113615) | the Formin/Diaphanous family which is involved in the |
| | cytoskeleton organization. |
| | Four interactors obtained from Muscle contain the same 0.6 kb |
| | fragment which matches the cDNA coding for the human |
| | FHOS protein. The insert start at position 2908 (frame 1) and |
| | covers the C-terminal domain [964-1164] of the FHOS protein |
| | (1164aa). |

Known S100B Interactions

| | |
|---|---|
| S100B (Genbank ID: J05600)-S100A9 | Fifty five interactors obtained from Liver (50 hits) and Muscle |
| (Genbank ID: M26311) | (5 hits) contain the same fragments which matches the cDNA |
| | coding for the human S100A9 protein. The inserts starts at |
| | position −21 (frame 3), adding 7 aa at the junction and should |
| | cover the entire coding region of S100A9 (114aa). |
| S100B (Genbank ID: J05600)- S100A6 | Five interactors obtained from Muscle contain a 0.4 kb |
| (Calcyclin)(Genbank ID: M14300) | fragment which matches the cDNA coding for the human |
| | S100A6 protein. Four insert start at position −221 (frame 2) and |
| | should cover the entire coding region of the S100A6 protein |
| | (94aa). One insert start at position −36 (frame 2) and covers the |
| | entire coding region of the S100A6 protein (94aa). However |
| | there is a natural stop codon (TAA) 43 bp before the ATG |
| | indicating that S100A6 is not expressed as a Ga14-AD fusion in |
| | four of the five clones. |

Confirmation and 1×1 Tests

To identify additional interactions between the baits and the prey, each bait was cloned in fusion with the Gal4-AD domain and each preys was cloned in fusion with the Gal4-BD domain.

To facilitate these DNA transfers, a new pAD plasmid was engineered to contain the same recombination sequences than the pBD plasmid to transfer any insert by gap-repair without PCR. The new plasmid was controlled by sequencing and was tested for its capacity to not affect known interactions.

To transfer the preys into the BD plasmid, the inserts from the AD plasmid were amplified and the PCR product was transferred into the BD plasmid by gap-repair. Four clones for each construct were controlled by PCR sizing and two positives were used for the mating experiments.

The six S100A1 and the eleven S100B interactions passed confirmation. Eleven out of 17 also confirmed in the opposite orientation. In addition, 5 interacting proteins interacted with both S100A1 and S100B: fibrinogen, RanBPM, S100B-IP4, NQO2, S100B-IP10 and FHOS (Table 8). Two interacting protein (FHOS and RanBPM) interacted with both full-length and truncated alleles of the baits. The other interacting proteins interacted exclusively with the full length (S100A6 and S100A9) or the truncated allele. These results indicate the importance of the C-terminal domain for the specificity of the S100 interactions.

TABLE 8
Interaction Between the S100A1 and S100B Interacting Proteins

| | | Binding Domain Fusions | | | | Activation Domain Fusions | | | |
|---|---|---|---|---|---|---|---|---|---|
| | PREYS | S100A1-1 | S100A1-2 | S100B-1 | S100B-2 | S100A1-1 | S100A1-2 | S100B-1 | S100B-2 |
| S100A1-IP | Fibrinogen | No | Yes | No | Yes | No | Yes | No | Yes |
| | Novel-IP2 | No | Yes | No | No | No | No | No | No |
| | RanBPM | Yes | Yes | No | Yes | No | No | No | No |
| | Novel-IP4 | No | Yes | No | No | No | Yes | No | No |
| | Profilin SV | No | Yes | No | No | No | Yes | No | No |
| | Novel-IP6 | No | Yes | No | No | No | No | No | No |
| S100B-IP | S100A9 | No | No | Yes | No | No | No | No | No |
| | Fibri. | No | Yes | No | Yes | No | Yes | No | Yes |
| | New-IP3 | No | No | No | Yes | No | No | No | Yes |
| | New-IP4 | No | Yes | No | Yes | No | No | No | Yes |
| | KIAA0629 | No | No | No | Yes | No | No | No | No |
| | ATPase | No | No | No | Yes | No | No | No | Yes |
| | Synphilin | No | No | No | Yes | ND | ND | ND | ND |
| | NQO2 | No | Yes | No | Yes | No | No | No | No |
| | S100A6 | No | No | Yes | No | No | No | No | No |
| | New-IP10 | No | No | No | Yes | No | No | No | Yes |
| | FHOS | Yes | Yes | Yes | Yes | No | No | No | No |

Interaction Generated by the S100A1 Interacting Proteins

When the six S100A1-interacting proteins were tested against each other (Table 9), 2 additional interactions (RanBPM with Novel-IP4 and Profilin) were identified.

When tested against other interacting proteins identified within the invention, 3 additional interactions for p53BP2 with three S100B interacting proteins: S100B-IP4, S100B-NOQ2, and S100B-IP10, were observed.

TABLE 9
Interactions Between the Proteins Interacting with S100A1

| AD | BD FUSIONS | | | | | |
|---|---|---|---|---|---|---|
| FUSIONS | Fibrinogen | Novel-IP2 | RanBPM | Novel-IP4 | Profilin SV | Novel-IP6 |
| Fibrinogen | ND | No | No | No | No | No |
| Novel-IP2 | ND | No | No | No | No | No |
| RanBPM | ND | No | No | No | No | No |
| Novel-IP4 | ND | No | Yes | No | No | No |
| Profilin SV | ND | No | Yes | No | No | No |
| Novel-IP6 | ND | No | No | No | No | No |

Interaction Generated by the S100B Interacting Proteins

When the eleven S100B interacting proteins were tested against each other (Table 10), 1 additional interaction (S100B-NOV9/S100B-NOV10), as well as one self interaction for NQO2, was identified.

TABLE 10
Interactions between the proteins binding to S100B

| AD | BD FUSIONS | | | | | | | | | | |
|---|---|---|---|---|---|---|---|---|---|---|---|
| FUSIONS | S100A9 | Fibri. | N-IP3 | N-IP4 | KIAA | ATPase | Synph | NQO2 | S100A6 | New-IP10 | FHOS |
| S100A9 | No | No | No | No | No | No | ND | No | No | No | No |
| Fibrinogen | No | No | No | No | No | No | ND | No | No | No | No |
| New-IP3 | No | No | No | No | No | No | ND | No | No | Yes | No |
| New-IP4 | No | No | Yes | No | No | No | ND | No | No | No | No |
| KIAA0629 | No | No | No | No | No | No | ND | No | No | No | No |
| ATPase | No | No | No | No | No | No | ND | No | No | Yes | No |
| Synphilin | No | No | No | No | No | No | ND | No | No | No | No |
| NQO2 | No | No | No | No | No | No | ND | Yes | No | No | No |
| S100A6 | No | No | No | No | No | No | ND | No | No | Yes | No |
| New-IP10 | No | No | No | No | No | No | ND | No | No | No | No |
| FHOS | No | No | No | No | No | No | ND | No | No | No | No |

Embodiments of these complexes containing the binding domains or conservative variants of these polypeptides are within the scope of the present invention, as are polypeptides which contain the polypeptides recited in Table 1.

Complexes Containing One or More Human Polypeptides

The invention also provides purified complexes of two or more human polypeptides. In some embodiments, the interacting polypeptides are human orthologs of the interacting yeast polypeptide.

In certain embodiments, one of the ortholog polypeptides includes a “bait” polypeptide selected from the polypeptides recited in Table 1, column 2, and the other ortholog polypeptide includes a “prey” protein selected from the polypeptides recited in Table 1, column 3. In some embodiments the first and second polypeptides of the complex are the polypeptides enumerated in Table 1. In some embodiments a first polypeptide is a “bait” polypeptide and a second polypeptide is “target” polypeptide, while in other embodiments the first polypeptide is a “target” polypeptide and the second is a “bait” polypeptide. Conservative variants of either polypeptide which retain binding specificity are within the scope of the invention, as are labeled forms of the complexes, as described above.

In other embodiments, the polypeptides are the binding domains of the “bait” and “prey” polypeptides listed in Table 1. A binding domain of a given first polypeptide may be any number of amino acids sufficient to specifically bind to, and complex with, the corresponding second polypeptide under conditions suitable for complex formation. A binding domain may be the minimal number of amino acids required to retain binding affinity, or may be a larger fragment or derivative of the polypeptides listed in Table 1, columns 2 and 3.

In certain embodiments the first and second polypeptides of the chimeric complex are the polypeptides recited in Table 1, columns 2 and 3. Conservative variants of the polypeptides which retain binding specificity are within the scope of the invention, as are labeled forms of the chimeric complexes, and chimeric complexes of binding domains, as described above.

NOVX Nucleic Acids

The nucleic acids of the invention include those that encode a NOVX polypeptide or protein. As used herein, the terms polypeptide and protein are interchangeable.

In some embodiments, a NOVX nucleic acid encodes a mature NOVX polypeptide. As used herein, a “mature” form of a polypeptide or protein described herein relates to the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an open reading frame described herein. The product “mature” form arises, again by way of nonlimiting example, as a result of one or more naturally occurring processing steps that may take place within the cell in which the gene product arises. Examples of such processing steps leading to a “mature” form of a polypeptide or protein include the cleavage of the N-terminal methionine residue encoded by the initiation codon of an open reading frame, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

Among the NOVX nucleic acids is the nucleic acid whose sequence is provided in SEQ ID NO: 1, 2, 3, 4, 5, 6, 7, 8, 9, 11, 12, or 13, or a fragment thereof. Additionally, the invention includes mutant or variant nucleic acids of SEQ ID NO: 1, 2, 3, 4, 5, 6, 7, 8, 9, 11, 12, 13, or a fragment thereof, any of whose bases may be changed from the corresponding bases shown in SEQ ID NO: 1, 2, 3, 4, 5, 6, 7, 8, 9, 11, 12, or 13, while still encoding a protein that maintains at least one of its NOVX-like activities and physiological functions (i.e., modulating angiogenesis, neuronal development). The invention further includes the complement of the nucleic acid sequence of SEQ ID NO: 1, 2, 3, 4, 5, 6, 7, 8, 9, 11, 12, or 13, including fragments, derivatives, analogs and homologs thereof. The invention additionally includes nucleic acids or nucleic acid fragments, or complements thereto, whose structures include chemical modifications.

One aspect of the invention pertains to isolated nucleic acid molecules that encode NOVX proteins or biologically active portions thereof. Also included are nucleic acid fragments sufficient for use as hybridization probes to identify NOVX-encoding nucleic acids (e.g., NOVX mRNA) and fragments for use as polymerase chain reaction (PCR) primers for the amplification or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but preferably is double-stranded DNA.

“Probes” refer to nucleic acid sequences of variable length, preferably between at least about 10 nucleotides (nt), 100 nt, or as many as about, e.g., 6,000 nt, depending on use. Probes are used in the detection of identical, similar, or complementary nucleic acid sequences. Longer length probes are usually obtained from a natural or recombinant source, are highly specific and much slower to hybridize than oligomers. Probes may be single- or double-stranded and designed to have specificity in PCR, membrane-based hybridization technologies, or ELISA-like technologies.

An “isolated” nucleic acid molecule is one that is separated from other nucleic acid molecules that are present in the natural source of the nucleic acid. Examples of isolated nucleic acid molecules include, but are not limited to, recombinant DNA molecules contained in a vector, recombinant DNA molecules maintained in a heterologous host cell, partially or substantially purified nucleic acid molecules, and synthetic DNA or RNA molecules. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated NOVX nucleic acid molecule can contain less than about 50 kb, 25 kb, 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the present invention, e.g., a nucleic acid molecule having the nucleotide sequence of SEQ ID NO: 1, 2, 3, 4, 5, 6, 7, 8, 9, 11, 12, 13, or a complement of any of this nucleotide sequence, can be isolated using standard molecular biology techniques and the sequence information provided herein. Using all or a portion of the nucleic acid sequence of SEQ ID NO: 1, 2, 3, 4, 5, 6, 7, 8, 9, 11, 12, or 13, as a hybridization probe, NOVX nucleic acid sequences can be isolated using standard hybridization and cloning techniques (e.g., as described in Sambrook et al., eds., MOLECULAR CLONING: A LABORATORY MANUAL 2^{nd} Ed., Cold Spring Harbor Laboratory Press, Cold Spring Harbor, N.Y., 1989; and Ausubel, et al., eds., CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment, an oligonucleotide comprising a nucleic acid molecule less than 100 nt in length would further comprise at lease 6 contiguous nucleotides of SEQ ID NO: 1, 2, 3, 4, 5, 6, 7, 8, 9, 11, 12, 13, or a complement thereof. Oligonucleotides may be chemically synthesized and may be used as probes.

In another embodiment, an isolated nucleic acid molecule of the invention comprises a nucleic acid molecule that is a complement of the nucleotide sequence shown in SEQ ID NO: 1, 2, 3, 4, 5, 6, 7, 8, 9, 11, 12, 13, or a portion of this nucleotide sequence. A nucleic acid molecule that is complementary to the nucleotide sequence shown in SEQ ID NO: 1, 2, 3, 4, 5, 6, 7, 8, 9, 11, 12, or 13 is one that is sufficiently complementary to the nucleotide sequence shown in SEQ ID NO: 1, 2, 3, 4, 5, 6, 7, 8, 9, 11, 12, or 13 that it can hydrogen bond with little or no mismatches to the nucleotide sequence shown in SEQ ID NO: 1, 2, 3, 4, 5, 6, 7, 8, 9, 11, 12, or 13, thereby forming a stable duplex.

As used herein, the term “complementary” refers to Watson-Crick or Hoogsteen base pairing between nucleotide units of a nucleic acid molecule, and the term “binding” means the physical or chemical interaction between two polypeptides or compounds or associated polypeptides or compounds or combinations thereof. Binding includes ionic, non-ionic, Von der Waals, hydrophobic interactions, etc. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Moreover, the nucleic acid molecule of the invention can comprise only a portion of the nucleic acid sequence of SEQ ID NO: 1, 2, 3, 4, 5, 6, 7, 8, 9, 11, 12, or 13, e.g., a fragment that can be used as a probe or primer, or a fragment encoding a biologically active portion of NOVX. Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, 85%, 90%, 95%, 98%, or even 99% identity (with a preferred identity of 80-99%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below. An exemplary program is the Gap program (Wisconsin Sequence Analysis Package, Version 8 for UNIX, Genetics Computer Group, University Research Park, Madison, Wis.) using the default settings, which uses the algorithm of Smith and Waterman (Adv. Appl. Math., 1981, 2: 482-489, which is incorporated herein by reference in its entirety).

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of a NOVX polypeptide. Isoforms can be expressed in different tissues of the same organism as a result of, for example, alternative splicing of RNA. Alternatively, isoforms can be encoded by different genes. In the present invention, homologous nucleotide sequences include nucleotide sequences encoding for a NOVX polypeptide of species other than humans, including, but not limited to, mammals, and thus can include, e.g., mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the nucleotide sequence encoding human NOVX protein. Homologous nucleic acid sequences include those nucleic acid sequences that encode conservative amino acid substitutions, as well as a polypeptide having NOVX activity. A homologous amino acid sequence does not encode the amino acid sequence of a human NOVX polypeptide.

The nucleotide sequence determined from the cloning of the human NOVX gene allows for the generation of probes and primers designed for use in identifying and/or cloning NOVX homologues in other cell types, e.g., from other tissues, as well as NOVX homologues from other mammals. The probe/primer typically comprises a substantially purified oligonucleotide. The oligonucleotide typically comprises a region of nucleotide sequence that hybridizes under stringent conditions to at least about 12, 25, 50, 100, 150, 200, 250, 300, 350 or 400 or more consecutive sense strand nucleotide sequence of SEQ ID NO: 1, 2, 3, 4, 5, 6, 7, 8, 9, 11, 12, or 13; or an anti-sense strand nucleotide sequence of SEQ ID NO: 1, 2, 3, 4, 5, 6, 7, 8, 9, 11, 12, or 13; or of a naturally occurring mutant of SEQ ID NO: 1, 2, 3, 4, 5, 6, 7, 8, 9, 11, 12, or 13.

Probes based on the human NOVX nucleotide sequence can be used to detect transcripts or genomic sequences encoding the same or homologous proteins. In various embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a NOVX protein, such as by measuring a level of a NOVX-encoding nucleic acid in a sample of cells from a subject e.g., detecting NOVX mRNA levels or determining whether a genomic NOVX gene has been mutated or deleted.

A “polypeptide having a biologically active portion of NOVX” refers to polypeptides exhibiting activity similar, but not necessarily identical to, an activity of a polypeptide of the present invention, including mature forms, as measured in a particular biological assay, with or without dose dependency. A nucleic acid fragment encoding a “biologically active portion of NOVX” can be prepared by isolating a portion of SEQ ID NO: 1, 2, 3, 4, 5, 6, 7, 8, 9, 11, 12, or 13 that encodes a polypeptide having a NOVX biological activity (biological activities of the NOVX proteins are described below), expressing the encoded portion of NOVX protein (e.g., by recombinant expression in vitro) and assessing the activity of the encoded portion of NOVX. For example, a nucleic acid fragment encoding a biologically active portion of NOVX can optionally include an ATP-binding domain. In another embodiment, a nucleic acid fragment encoding a biologically active portion of NOVX includes one or more regions.

NOVX Variants

The invention further encompasses nucleic acid molecules that differ from the nucleotide sequences shown in SEQ ID NO: 1, 2, 3, 4, 5, 6, 7, 8, 9, 11, 12, or 13 due to the degeneracy of the genetic code. These nucleic acids thus encode the same NOVX protein as that encoded by the nucleotide sequence shown in SEQ ID NO: 1, 2, 3, 4, 5, 6, 7, 8, 9, 11, 12, or 13.

In addition to the human NOVX nucleotide sequence shown in SEQ ID NO: 1, 2, 3, 4, 5, 6, 7, 8, 9, 11, 12, or 13, it will be appreciated by those skilled in the art that DNA sequence polymorphisms that lead to changes in the amino acid sequences of NOVX may exist within a population (e.g., the human population). Such genetic polymorphism in the NOVX gene may exist among individuals within a population due to natural allelic variation. As used herein, the terms “gene” and “recombinant gene” refer to nucleic acid molecules comprising an open reading frame encoding a NOVX protein, preferably a mammalian NOVX protein. Such natural allelic variations can typically result in 1-5% variance in the nucleotide sequence of the NOVX gene. Any and all such nucleotide variations and resulting amino acid polymorphisms in NOVX that are the result of natural allelic variation and that do not alter the functional activity of NOVX are intended to be within the scope of the invention.

Moreover, nucleic acid molecules encoding NOVX proteins from other species, and thus that have a nucleotide sequence that differs from the human sequence of SEQ ID NO: 1, 2, 3, 4, 5, 6, 7, 8, 9, 11, 12, or 13 are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions. For example, a soluble human NOVX cDNA can be isolated based on its homology to human membrane-bound NOVX. Likewise, a membrane-bound human NOVX cDNA can be isolated based on its homology to soluble human NOVX.

Accordingly, in another embodiment, an isolated nucleic acid molecule of the invention is at least 6 nucleotides in length and hybridizes under stringent conditions to the nucleic acid molecule comprising the nucleotide sequence of SEQ ID NO: 1, 2, 3, 4, 5, 6, 7, 8, 9, 11, 12, or 13. In another embodiment, the nucleic acid is at least 10, 25, 50, 100, 250, 500 or 750 nucleotides in length. In another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tₘ) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions is hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C. This hybridization is followed by one or more washes in 0.2×SSC, 0.01% BSA at 50° C. An isolated nucleic acid molecule of the invention that hybridizes under stringent conditions to the sequence of SEQ ID NO: 1, 2, 3, 4, 5, 6, 7, 8, 9, 10, 11, 12, or 13 corresponds to a naturally occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NO: 1, 2, 3, 4, 5, 6, 7, 8, 9, 11, 12, or 13, or fragments, analogs or derivatives thereof, under conditions of moderate stringency is provided. A non-limiting example of moderate stringency hybridization conditions are hybridization in 6×SSC, 5× Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well known in the art. See, e.g., Ausubel et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY.

In a third embodiment, a nucleic acid that is hybridizable to the nucleic acid molecule comprising the nucleotide sequence of SEQ ID NO: 1, 2, 3, 4, 5, 6, 7, 8, 9, 11, 12, or 13, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981, Proc Natl Acad Sci USA 78: 6789-6792.

Conservative Mutations

In addition to naturally-occurring allelic variants of the NOVX sequence that may exist in the population, the skilled artisan will further appreciate that changes can be introduced by mutation into the nucleotide sequence of SEQ ID NO: 1, 2, 3, 4, 5, 6, 7, 8, 9, 11, 12, or 13, thereby leading to changes in the amino acid sequence of the encoded NOVX protein, without altering the functional ability of the NOVX protein. For example, nucleotide substitutions leading to amino acid substitutions at “non-essential” amino acid residues can be made in the sequence of SEQ ID NO: 1, 2, 3, 4, 5, 6, 7, 8, 9, 11, 12, or 13. A “non-essential” amino acid residue is a residue that can be altered from the wild-type sequence of NOVX without altering the biological activity, whereas an “essential” amino acid residue is required for biological activity. For example, amino acid residues that are conserved among the NOVX proteins of the present invention, are predicted to be particularly unamenable to alteration.

Another aspect of the invention pertains to nucleic acid molecules encoding NOVX proteins that contain changes in amino acid residues that are not essential for activity. Such NOVX proteins differ in amino acid sequence from the polypeptide encoded by the nucleotide sequence of SEQ ID NO: 1, 2, 3, 4, 5, 6, 7, 8, 9, 11, 12, or 13 yet retain biological activity. In one embodiment, the isolated nucleic acid molecule comprises a nucleotide sequence encoding a protein, wherein the protein comprises an amino acid sequence at least about 75% homologous to the amino acid sequence from the polypeptide encoded by the nucleotide sequence of SEQ ID NO: 1, 2, 3, 4, 5, 6, 7, 8, 9, 11, 12, or 13. Preferably, the protein encoded by the nucleic acid is at least about 80% homologous to the polypeptide encoded by the nucleotide sequence of SEQ ID NO: 1, 2, 3, 4, 5, 6, 7, 8, 9, 11, 12, or 13, more preferably at least about 90%, 95%, 98%, and most preferably at least about 99% homologous to the polypeptide encoded by the nucleotide sequence of SEQ ID NO: 1, 2, 3, 4, 5, 6, 7, 8, 9, 11, 12, or 13.

An isolated nucleic acid molecule encoding a NOVX protein homologous to the protein of can be created by introducing one or more nucleotide substitutions, additions or deletions into the nucleotide sequence of SEQ ID NO: 1, 2, 3, 4, 5, 6, 7, 8, 9, 11, 12, or 13, such that one or more amino acid substitutions, additions or deletions are introduced into the encoded protein.

Mutations can be introduced into the nucleotide sequence of SEQ ID NO: 1, 2, 3, 4, 5, 6, 7, 8, 9, 11, 12, or 13 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in NOVX is replaced with another amino acid residue from the same side chain family. Alternatively, in another embodiment, mutations can be introduced randomly along all or part of a NOVX coding sequence, such as by saturation mutagenesis, and the resultant mutants can be screened for NOVX biological activity to identify mutants that retain activity. Following mutagenesis of SEQ ID NO: 1, 2, 3, 4, 5, 6, 7, 8, 9, 11, 12, or 13 the encoded protein can be expressed by any recombinant technology known in the art and the activity of the protein can be determined.

In one embodiment, a mutant NOVX protein can be assayed for (1) the ability to form protein:protein interactions with other NOVX proteins, other cell-surface proteins, or biologically active portions thereof, (2) complex formation between a mutant NOVX protein and a NOVX receptor; (3) the ability of a mutant NOVX protein to bind to an intracellular target protein or biologically active portion thereof; (e.g., avidin proteins); (4) the ability to bind NOVX protein; or (5) the ability to specifically bind an anti-NOVX protein antibody.

Antisense NOVX Nucleic Acids

Another aspect of the invention pertains to isolated antisense nucleic acid molecules that are hybridizable to or complementary to the nucleic acid molecule comprising the nucleotide sequence of SEQ ID NO: 1, 2, 3, 4, 5, 6, 7, 8, 9, 11, 12, or 13, or fragments, analogs or derivatives thereof. An “antisense” nucleic acid comprises a nucleotide sequence that is complementary to a “sense” nucleic acid encoding a protein, e.g., complementary to the coding strand of a double-stranded cDNA molecule or complementary to an mRNA sequence. In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of a NOVX protein encoded by SEQ ID NO: 1, 2, 3, 4, 5, 6, 7, 8, 9, 11, 12, or 13 or antisense nucleic acids complementary to a NOVX nucleic acid sequence of SEQ ID NO: 1, 2, 3, 4, 5, 6, 7, 8, 9, 11, 12, or 13 are additionally provided.

In one embodiment, an antisense nucleic acid molecule is antisense to a “coding region” of the coding strand of a nucleotide sequence encoding NOVX. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding NOVX. The term “noncoding region” refers to 5′ and 3′ sequences, which flank the coding region that are not translated into amino acids (i.e., also referred to as 5′ and 3′ untranslated regions).

Given the coding strand sequences encoding NOVX disclosed herein (e.g., SEQ ID NO: 1, 2, 3, 4, 5, 6, 7, 8, 9, 11, 12, or 13), antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a NOVX protein to thereby inhibit expression of the protein, e.g., by inhibiting transcription and/or translation. The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids Res 15: 6625-6641). The antisense nucleic acid molecule can also comprise a 2′-o-methylribonucleotide (Inoue et al. (1987) Nucleic Acids Res 15: 6131-6148) or a chimeric RNA-DNA analogue (Inoue et al. (1987) FEBS Lett 215: 327-330).

Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

NOVX Ribozymes and PNA Moieties

In still another embodiment, an antisense nucleic acid of the invention is a ribozyme. Ribozymes are catalytic RNA molecules with ribonuclease activity that are capable of cleaving a single-stranded nucleic acid, such as a mRNA, to which they have a complementary region. Thus, ribozymes (e.g., hammerhead ribozymes (described in Haselhoff and Gerlach (1988) Nature 334:585-591)) can be used to catalytically cleave NOVX mRNA transcripts to thereby inhibit translation of NOVX mRNA. A ribozyme having specificity for a NOVX-encoding nucleic acid can be designed based upon the nucleotide sequence of a NOVX DNA disclosed herein (i.e., SEQ ID NO: 1, 2, 3, 4, 5, 6, 7, 8, 9, 11, 12, or 13). For example, a derivative of a Tetrahymena L-19 IVS RNA can be constructed in which the nucleotide sequence of the active site is complementary to the nucleotide sequence to be cleaved in a NOVX-encoding mRNA. See, e.g., Cech et al. U.S. Pat. No. 4,987,071; and Cech et al. U.S. Pat. No. 5,116,742. Alternatively, NOVX mRNA can be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX (e.g., the NOVX promoter and/or enhancers) to form triple helical structures that prevent transcription of the NOVX gene in target cells. See generally, Helene. (1991) Anticancer Drug Des. 6: 569-84; Helene. et al. (1992) Ann. N.Y. Acad. Sci. 660:27-36; and Maher (1992) Bioassays 14: 807-15.

In various embodiments, the nucleic acids of NOVX can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorg Med Chem 4: 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) above; Perry-O'Keefe et al. (1996) PNAS 93: 14670-675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, e.g., in the analysis of single base pair mutations in a gene by, e.g., PNA directed PCR clamping; as artificial restriction enzymes when used in combination with other enzymes, e.g., S1 nucleases (Hyrup B. (1996) above); or as probes or primers for DNA sequence and hybridization (Hyrup et al. (1996), above; Perry-O'Keefe (1996), above).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996) above). The synthesis of PNA-DNA chimeras can be performed as described in Hyrup (1996) above and Finn et al. (1996) Nucl Acids Res 24: 3357-63. For example, a DNA chain can be synthesized on a solid support using standard phosphoramidite coupling chemistry, and modified nucleoside analogs, e.g., 5′-(4-methoxytrityl) amino-5′-deoxy-thymidine phosphoramidite, can be used between the PNA and the 5′ end of DNA (Mag et al. (1989) Nucl Acid Res 17: 5973-88). PNA monomers are then coupled in a stepwise manner to produce a chimeric molecule with a 5′ PNA segment and a 3′ DNA segment (Finn et al. (1996) above). Alternatively, chimeric molecules can be synthesized with a 5′ DNA segment and a 3′ PNA segment. See, Petersen et al. (1975) Bioorg Med Chem Lett 5: 1119-11124.

In other embodiments, the oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810) or the blood-brain barrier (see, e.g., PCT Publication No. WO89/10134). In addition, oligonucleotides can be modified with hybridization triggered cleavage agents (See, e.g., Krol et al., 1988, BioTechniques 6:958-976) or intercalating agents. (See, e.g., Zon, 1988, Pharm. Res. 5: 539-549). To this end, the oligonucleotide may be conjugated to another molecule, e.g., a peptide, a hybridization triggered cross-linking agent, a transport agent, a hybridization-triggered cleavage agent, etc.

NOVX Polypeptides

A NOVX polypeptide of the invention includes the NOVX-like protein encoded by SEQ ID NO: 1, 2, 3, 4, 5, 6, 7, 8, 9, 11, 12, or 13. The invention also includes a mutant or variant protein any of whose residues may be changed from the corresponding residue, while still encoding a protein that maintains its NOVX-like activities and physiological functions, or a functional fragment thereof. In some embodiments, up to 20% or more of the residues may be so changed in the mutant or variant protein. In some embodiments, the NOVX polypeptide according to the invention is a mature polypeptide.

In general, a NOVX-like variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, a NOVX protein or polypeptide can be synthesized chemically using standard peptide synthesis techniques.

An “isolated” or “purified” protein or biologically active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX protein in which the protein is separated from cellular components of the cells from which it is isolated or recombinantly produced. In one embodiment, the language “substantially free of cellular material” includes preparations of NOVX protein having less than about 30% (by dry weight) of non-NOVX protein (also referred to herein as a “contaminating protein”), more preferably less than about 20% of non-NOVX protein, still more preferably less than about 10% of non-NOVX protein, and most preferably less than about 5% non-NOVX protein. When the NOVX protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of NOVX protein in which the protein is separated from chemical precursors or other chemicals that are involved in the synthesis of the protein. In one embodiment, the language “substantially free of chemical precursors or other chemicals” includes preparations of NOVX protein having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically active portions of a NOVX protein include peptides comprising amino acid sequences sufficiently homologous to or derived from the amino acid sequence of the NOVX protein, that include fewer amino acids than the full length NOVX proteins, and exhibit at least one activity of a NOVX protein. Typically, biologically active portions comprise a domain or motif with at least one activity of the NOVX protein. A biologically active portion of a NOVX protein can be a polypeptide, which is, for example, 10, 25, 50, 100 or more amino acids in length.

A biologically active portion of a NOVX protein of the present invention may contain at least one of the above-identified domains conserved between the NOVX proteins, e.g. TSR modules. Moreover, other biologically active portions, in which other regions of the protein are deleted, can be prepared by recombinant techniques and evaluated for one or more of the functional activities of a native NOVX protein.

In an embodiment, the NOVX protein has an amino acid sequence encoded by SEQ ID NO: 1, 2, 3, 4, 5, 6, 7, 8, 9, 11, 12, or 13. In other embodiments, the NOVX protein is substantially homologous to the polypeptide encoded by SEQ ID NO: 1, 2, 3, 4, 5, 6, 7, 8, 9, 11, 12, or 13 and retains the functional activity of the protein encoded by SEQ ID NO: 1, 2, 3, 4, 5, 6, 7, 8, 9, 11, 12, or 13 yet differs in amino acid sequence due to natural allelic variation or mutagenesis, as described in detail below. Accordingly, in another embodiment, the NOVX protein is a protein that comprises an amino acid sequence at least about 45% homologous to the amino acid sequence encoded by SEQ ID NO: 1, 2, 3, 4, 5, 6, 7, 8, 9, 11, 12, or 13 and retains the functional activity of the NOVX proteins encoded by SEQ ID NO: 1, 2, 3, 4, 5, 6, 7, 8, 9, 11, 12, or 13.

Determining Homology Between Two or More Sequence

To determine the percent homology of two amino acid sequences or of two nucleic acids, the sequences are aligned for optimal comparison purposes (e.g., gaps can be introduced in either of the sequences being compared for optimal alignment between the sequences). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch 1970 J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NO: 1, 2, 3, 4, 5, 6, 7, 8, 9, 11, 12, or 13.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region. The term “percentage of positive residues” is calculated by comparing two optimally aligned sequences over that region of comparison, determining the number of positions at which the identical and conservative amino acid substitutions, as defined above, occur in both sequences to yield the number of matched positions, dividing the number of matched positions by the total number of positions in the region of comparison (i.e., the window size), and multiplying the result by 100 to yield the percentage of positive residues.

Chimeric and Fusion Proteins

The invention also provides NOVX chimeric or fusion proteins. As used herein, a NOVX “chimeric protein” or “fusion protein” comprises a NOVX polypeptide operatively linked to a non-NOVX polypeptide. An “NOVX polypeptide” refers to a polypeptide having an amino acid sequence corresponding to NOVX, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within a NOVX fusion protein the NOVX polypeptide can correspond to all or a portion of a NOVX protein. In one embodiment, a NOVX fusion protein comprises at least one biologically active portion of a NOVX protein. In another embodiment, a NOVX fusion protein comprises at least two biologically active portions of a NOVX protein. Within the fusion protein, the term “operatively linked” is intended to indicate that the NOVX polypeptide and the non-NOVX polypeptide are fused in-frame to each other. The non-NOVX polypeptide can be fused to the N-terminus or C-terminus of the NOVX polypeptide.

For example, in one embodiment a NOVX fusion protein comprises a NOVX polypeptide operably linked to the extracellular domain of a second protein. Such fusion proteins can be further utilized in screening assays for compounds that modulate NOVX activity (such assays are described in detail below).

In another embodiment, the fusion protein is a GST-NOVX fusion protein in which the NOVX sequences are fused to the C-terminus of the GST (i.e., glutathione S-transferase) sequences. Such fusion proteins can facilitate the purification of recombinant NOVX.

In another embodiment, the fusion protein is a NOVX-immunoglobulin fusion protein in which the NOVX sequences comprising one or more domains are fused to sequences derived from a member of the immunoglobulin protein family. The NOVX-immunoglobulin fusion proteins of the invention can be incorporated into pharmaceutical compositions and administered to a subject to inhibit an interaction between a NOVX ligand and a NOVX protein on the surface of a cell, to thereby suppress NOVX-mediated signal transduction in vivo. In one nonlimiting example, a contemplated NOVX ligand of the invention is the NOVX receptor. The NOVX-immunoglobulin fusion proteins can be used to affect the bioavailability of a NOVX cognate ligand. Inhibition of the NOVX ligand/NOVX interaction may be useful therapeutically for both the treatment of proliferative and differentiative disorders, e,g., cancer as well as modulating (e.g., promoting or inhibiting) cell survival, as well as acute and chronic inflammatory disorders and hyperplastic wound healing, e.g. hypertrophic scars and keloids. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with a NOVX ligand.

A NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Ausubel et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A NOVX-encoding nucleic acid can be cloned into such an expression vector such that the fusion moiety is linked in-frame to the NOVX protein.

NOVX Agonists and Antagonists

The present invention also pertains to variants of the NOVX proteins that function as either NOVX agonists (mimetics) or as NOVX antagonists. Variants of the NOVX protein can be generated by mutagenesis, e.g., discrete point mutation or truncation of the NOVX protein. An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX protein that function as either NOVX agonists (mimetics) or as NOVX antagonists can be identified by screening combinatorial libraries of mutants, e.g., truncation mutants, of the NOVX protein for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are known in the art (see, e.g., Narang (1983) Tetrahedron 39:3; Itakura et al. (1984) Annu Rev Biochem 53:323; Itakura et al. (1984) Science 198:1056; Ike et al. (1983) Nucl Acid Res 11:477.

Polypeptide Libraries

In addition, libraries of fragments of the NOVX protein coding sequence can be used to generate a variegated population of NOVX fragments for screening and subsequent selection of variants of a NOVX protein. In one embodiment, a library of coding sequence fragments can be generated by treating a double stranded PCR fragment of a NOVX coding sequence with a nuclease under conditions wherein nicking occurs only about once per molecule, denaturing the double stranded DNA, renaturing the DNA to form double stranded DNA that can include sense/antisense pairs from different nicked products, removing single stranded portions from reformed duplexes by treatment with S1 nuclease, and ligating the resulting fragment library into an expression vector. By this method, an expression library can be derived which encodes N-terminal and internal fragments of various sizes of the NOVX protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recrusive ensemble mutagenesis (REM), a new technique that enhances the frequency of functional mutants in the libraries, can be used in combination with the screening assays to identify NOVX variants (Arkin and Yourvan (1992) PNAS 89:7811-7815; Delgrave et al. (1993) Protein Engineering 6:327-331).

NOVX Antibodies

Also included in the invention are antibodies to NOVX proteins, or fragments of NOVX proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated NOVX-related protein of the invention may be intended to serve as an antigen, or a portion or fragment thereof, and additionally can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein, such as an amino acid sequence encoded by SEQ ID NO: 1, 2, 3, 4, 5, 6, 7, 8, 9, 11, 12, or 13, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX-related protein that is located on the surface of the protein, e.g., a hydrophilic region. A hydrophobicity analysis of the human NOVX-related protein sequence will indicate which regions of a NOVX-related protein are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each of which is incorporated herein by reference in its entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies relate to antibody molecules in which essentially the entire sequences of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: Monoclonal Antibodies and Cancer Therapy, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: Monoclonal Antibodies and Cancer Therapy, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al,(Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an Fab fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., 1991 EMBO J., 10:3655-3659.

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

NOVX Recombinant Expression Vectors and Host Cells

Another aspect of the invention pertains to vectors, preferably expression vectors, containing a nucleic acid encoding a NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews—Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as human, Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. Sequences including SEQ ID NO: 1, 2, 3, 4, 5, 6, 7, 8, 9, 11, 12, or 13 can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of a NOVX gene into which a deletion, addition or substitution has been introduced to thereby alter, e.g., functionally disrupt, the NOVX gene. The NOVX gene can be a human gene (e.g., the DNA of SEQ ID NO: 1, 2, 3, 4, 5, 6, 7, 8, 9, 11, 12, or 13), but more preferably, is a non-human homologue of a human NOVX gene. For example, a mouse homologue of human NOVX gene of SEQ ID NO: 1, 2, 3, 4, 5, 6, 7, 8, 9, 11, 12, or 13 can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl. Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction. A chemotherapeutic agent (such as Doxorubicin) is optionally contained within the liposome. See Gabizon et al., J. National Cancer Inst., 81(19): 1484 (1989).

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

Antibodies specifically binding a protein of the invention, as well as other molecules identified by the screening assays disclosed herein, can be administered for the treatment of various disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York. If the antigenic protein is intracellular and whole antibodies are used as inhibitors, internalizing antibodies are preferred. However, liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., 1993 Proc. Natl. Acad. Sci. USA, 90: 7889-7893. The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended. The active ingredients can also be entrapped in microcapsules prepared, for example, by coacervation techniques or by interfacial polymerization, for example, hydroxymethylcellulose or gelatin-microcapsules and poly(methylmethacrylate) microcapsules, respectively, in colloidal drug delivery systems (for example, liposomes, albumin microspheres, microemulsions, nano-particles, and nanocapsules) or in macroemulsions.

The formulations to be used for iv vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in a NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein. In addition, the anti-NOVX antibodies of the invention can be used to detect and isolate NOVX proteins and modulate NOVX activity. For example, NOVX activity includes growth and differentiation, antibody production, and tumor growth.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of a NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12:145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390, Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to a NOVX protein determined. The cell, for example, can be of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with a NOVX protein, wherein determining the ability of the test compound to interact with a NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with a NOVX target molecule. As used herein, a “target molecule” is a molecule with which a NOVX protein binds or interacts in nature, for example, a molecule on the surface of a cell which expresses a NOVX interacting protein, a molecule on the surface of a second cell, a molecule in the extracellular milieu, a molecule associated with the internal surface of a cell membrane or a cytoplasmic molecule. A NOVX target molecule can be a non-NOVX molecule or a NOVX protein or polypeptide of the invention In one embodiment, a NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with a NOVX target molecule can be accomplished by one of the methods described above for determining direct binding. In one embodiment, determining the ability of the NOVX protein to bind to or interact with a NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising a NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting a NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with a NOVX protein, wherein determining the ability of the test compound to interact with a NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to a NOVX target molecule by one of the methods described above for determining direct binding. In an alternative embodiment, determining the ability of the test compound to modulate the activity of NOVX protein can be accomplished by determining the ability of the NOVX protein further modulate a NOVX target molecule. For example, the catalytic/enzymatic activity of the target molecule on an appropriate substrate can be determined as described above.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with a NOVX protein, wherein determining the ability of the test compound to interact with a NOVX protein comprises determining the ability of the NOVX protein to preferentially bind to or modulate the activity of a NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl-N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also likely to be involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) identify an individual from a minute biological sample (tissue typing); and (ii) aid in forensic identification of a biological sample. Some of these applications are described in the subsections, below.

Tissue Typing

The NOVX sequences of the invention can be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NO: 1, 2, 3, 4, 5, 6, 7, 8, 9, 11, 12, or 13 are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. Disorders associated with aberrant NOVX expression of activity include, for example, disorders of renal and pancreatic dysfunction, e.g. diabetes, hypertension, cirrhosis, and cancer. The invention also provides for prognostic (or predictive) assays for determining whether an individual is at risk of developing a disorder associated with NOVX protein, nucleic acid expression or activity. For example, mutations in a NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.) Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NO: 1, 2, 3, 4, 5, 6, 7, 8, 9, 11, 12, or 13, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

One agent for detecting NOVX protein is an antibody capable of binding to NOVX protein, preferably an antibody with a detectable label. Antibodies directed against a protein of the invention may be used in methods known within the art relating to the localization and/or quantitation of the protein (e.g., for use in measuring levels of the protein within appropriate physiological samples, for use in diagnostic methods, for use in imaging the protein, and the like). In a given embodiment, antibodies against the proteins, or derivatives, fragments, analogs or homologs thereof, that contain the antigen binding domain, are utilized as pharmacologically-active compounds.

An antibody specific for a protein of the invention can be used to isolate the protein by standard techniques, such as immunoaffinity chromatography or immunoprecipitation. Such an antibody can facilitate the purification of the natural protein antigen from cells and of recombinantly produced antigen expressed in host cells. Moreover, such an antibody can be used to detect the antigenic protein (e.g., in a cellular lysate or cell supernatant) in order to evaluate the abundance and pattern of expression of the antigenic protein. Antibodies directed against the protein can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ³¹I, ³⁵S or ³H.

Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In one embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Such disorders include for example, disorders of renal and pancreas dysfunction, e.g. diabetes, hypertension, cirrhosis, and cancer.

Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in a NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding a NOVX-protein, or the misexpression of the NOVX gene. For example, such genetic lesions can be detected by ascertaining the existence of at least one of: (i) a deletion of one or more nucleotides from a NOVX gene; (ii) an addition of one or more nucleotides to a NOVX gene; (iii) a substitution of one or more nucleotides of a NOVX gene, (iv) a chromosomal rearrangement of a NOVX gene; (v) an alteration in the level of a messenger RNA transcript of a NOVX gene, (vi) aberrant modification of a NOVX gene, such as of the methylation pattern of the genomic DNA, (vii) the presence of a non-wild-type splicing pattern of a messenger RNA transcript of a NOVX gene, (viii) a non-wild-type level of a NOVX protein, (ix) allelic loss of a NOVX gene, and (x) inappropriate post-translational modification of a NOVX protein. As described herein, there are a large number of assay techniques known in the art which can be used for detecting lesions in a NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to a NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on a NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders (e.g. disorders of of renal and pancreas dysfunction, e.g. diabetes, hypertension, cirrhosis, and cancer). In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of a NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. Disorders associated with aberrant NOVX expression include, for example, disorders of renal and pancreas dysfunction, e.g. diabetes, hypertension, cirrhosis, and cancer.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, a NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of a NOVX protein, a peptide, a NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of a NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering a NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated). Another example of such a situation is where the subject has an immunodeficiency disease (e.g., AIDS).

Antibodies of the invention, including polyclonal, monoclonal, humanized and fully human antibodies, may used as therapeutic agents. Such agents will generally be employed to treat or prevent a disease or pathology in a subject. An antibody preparation, preferably one having high specificity and high affinity for its target antigen, is administered to the subject and will generally have an effect due to its binding with the target. Such an effect may be one of two kinds, depending on the specific nature of the interaction between the given antibody molecule and the target antigen in question. In the first instance, administration of the antibody may abrogate or inhibit the binding of the target with an endogenous ligand to which it naturally binds. In this case, the antibody binds to the target and masks a binding site of the naturally occurring ligand, wherein the ligand serves as an effector molecule. Thus the receptor mediates a signal transduction pathway for which ligand is responsible.

Alternatively, the effect may be one in which the antibody elicits a physiological result by virtue of binding to an effector binding site on the target molecule. In this case the target, a receptor having an endogenous ligand which may be absent or defective in the disease or pathology, binds the antibody as a surrogate effector ligand, initiating a receptor-based signal transduction event by the receptor.

A therapeutically effective amount of an antibody of the invention relates generally to the amount needed to achieve a therapeutic objective. As noted above, this may be a binding interaction between the antibody and its target antigen that, in certain cases, interferes with the functioning of the target, and in other cases, promotes a physiological response. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week.

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Chimeric Polypeptides, DNA, Vectors and Recombinant Cells

In a further aspect, the invention provides a chimeric polypeptide that includes sequences of two interacting proteins according to the invention. The interacting proteins can be, e.g., the interacting protein pairs disclosed in Table 1, herein. Also included are chimeric polypeptides including multimers, i.e., sequences from two or more pairs of interacting proteins. An example of such a chimeric polypeptide is a polypeptide that includes amino acid sequences from Protein Pair ID:1, and from Protein Pair ID:2. The chimeric polypeptide includes a region of a first protein covalently linked, e.g. via peptide bond, to a region of a second protein. In certain embodiments, the second protein is a species ortholog of the first protein. In some embodiments, the chimeric polypeptide contains regions of first and second proteins from yeast, where the proteins are selected from the “bait” and corresponding “prey” proteins recited in Table 1, columns 2 and 3, respectively.

In some embodiments, the chimeric polypeptide(s) of the complex include(s) six or more amino acids of a first protein covalently linked to six or more amino acids of a second protein. In other embodiments, the chimeric polypeptide includes at least one binding domain of a first or second protein.

Preferably, the chimeric polypeptide includes a region of amino acids of the first polypeptide able to bind to a second polypeptide. Alternatively, or in addition, the chimeric polypeptide includes a region of amino acids of the second polypeptide able to bind to the first polypeptide.

Nucleic acid encoding the chimeric polypeptide, as well as vectors and cells containing these nucleic acids, are within the scope of the present invention. The chimeric polypeptides can be constructed by expressing nucleic acids encoding chimeric polypeptides using recombinant methods, described above, then recovering the chimeric polypeptides, or by chemically synthesizing the chimeric polypeptides. Host-vector systems that can be used to express chimeric polypeptides include, e.g.: (i) mammalian cell systems which are infected with vaccinia virus, adenovirus; (ii) insect cell systems infected with baculovirus; (iii) yeast containing yeast vectors or (iv) bacteria transformed with bacteriophage, DNA, plasmid DNA, or cosmid DNA. Depending upon the host-vector system utilized, any one of a number of suitable transcription and translation elements may be used.

The expression of the specific proteins may be controlled by any promoter/enhancer known in the art including, e.g.: (i) the SV40 early promoter (see e.g., Bernoist & Chambon, Nature 290: 304-310 (1981)); (ii) the promoter contained within the 3′-terminus long terminal repeat of Rous Sarcoma Virus (see e.g., Yamamoto, et al., Cell 22: 787-797 (1980)); (iii) the Herpesvirus thymidine kinase promoter (see e.g., Wagner, et al., Proc. Natl. Acad. Sci. USA 78: 1441-1445 (1981)); (iv) the regulatory sequences of the metallothionein gene (see e.g., Brinster, et al., Nature 296: 39-42 (1982)); (v) prokaryotic expression vectors such as the β-lactamase promoter (see e.g., Villa-Kamaroff, et al., Proc. Natl. Acad. Sci. USA 75: 3727-3731 (1978)); (vi) the tac promoter (see e.g., DeBoer, et al, Proc. Natl. Acad. Sci. USA 80: 21-25 (1983)).

Plant promoter/enhancer sequences within plant expression vectors may also be utilized including, e.g.,: (i) the nopaline synthetase promoter (see e.g., Herrar-Estrella, et al., Nature 303: 209-213 (1984)); (ii) the cauliflower mosaic virus 35S RNA promoter (see e.g., Garder, et al., Nuc. Acids Res. 9: 2871 (1981)) and (iii) the promoter of the photosynthetic enzyme ribulose bisphosphate carboxylase (see e.g., Herrera-Estrella, et al., Nature 310: 115-120 (1984)).

Promoter/enhancer elements from yeast and other fungi (e.g., the Gal4 promoter, the alcohol dehydrogenase promoter, the phosphoglycerol kinase promoter, the alkaline phosphatase promoter), as well as the following animal transcriptional control regions, which possess tissue specificity and have been used in transgenic animals, may be utilized in the production of proteins of the present invention.

Other animal transcriptional control sequences derived from animals include, e.g.,: (i) the insulin gene control region active within pancreatic β-cells (see e.g., Hanahan, et al., Nature 315: 115-122 (1985)); (ii) the immunoglobulin gene control region active within lymphoid cells (see e.g., Grosschedl, et al., Cell 38: 647-658 (1984)); (iii) the albumin gene control region active within liver (see e.g., Pinckert, et al., Genes and Devel. 1: 268-276 (1987)); (iv) the myelin basic protein gene control region active within brain oligodendrocyte cells (see e.g., Readhead, et al., Cell 48: 703-712 (1987)); and (v) the gonadotrophin-releasing hormone gene control region active within the hypothalamus (see e.g., Mason, et al., Science 234: 1372-1378 (1986)).

The vector may include a promoter operably-linked to nucleic acid sequences which encode a chimeric polypeptide, one or more origins of replication, and optionally, one or more selectable markers (e.g., an antibiotic resistance gene). A host cell strain may be selected which modulates the expression of chimeric sequences, or modifies/processes the expressed proteins in a desired manner. Moreover, different host cells possess characteristic and specific mechanisms for the translational and post-translational processing and modification (e.g., glycosylation, phosphorylation, and the like) of expressed proteins. Appropriate cell lines or host systems may thus be chosen to ensure the desired modification and processing of the foreign protein is achieved. For example, protein expression within a bacterial system can be used to produce an unglycosylated core protein; whereas expression within mammalian cells ensures “native” glycosylation of a heterologous protein.

Antibodies Specific for Polypeptide Complexes

The invention further provides antibodies and antibody fragments (such as Fab or (Fab)2 fragments) that bind specifically to the complexes described herein. By “specifically binds” is meant an antibody that recognizes and binds to a particular polypeptide complex of the invention, but which does not substantially recognize or bind to other molecules in a sample, or to any of the polypeptides of the complex when those polypeptides are not complexed.

For example, a purified complex, or a portion, variant, or fragment thereof, can be used as an immunogen to generate antibodies that specifically bind the complex using standard techniques for polyclonal and monoclonal antibody preparation.

A full-length polypeptide complex can be used, if desired. Alternatively, the invention provides antigenic fragments of polypeptide complexes for use as immunogens. In some embodiments, the antigenic complex fragment includes at least 6, 8, 10, 15, 20, or 30 or more amino acid residues of a polypeptide. In one embodiment, epitopes encompassed by the antigenic peptide include the binding domains of the polypeptides, or are located on the surface of the protein, e.g., hydrophilic regions.

If desired, peptides containing antigenic regions can be selected using hydropathy plots showing regions of hydrophilicity and hydrophobicity. These plots may be generated by any method well known in the art, including, for example, the Kyte Doolittle or the Hopp Woods methods, either with or without Fourier transformation. See, e.g., Hopp and Woods, Proc. Nat. Acad. Sci. USA 78:3824-3828 (1981); Kyte and Doolittle, J. Mol. Biol. 157:105-142 (1982).

The term “antibody” as used herein refers to immunoglobulin molecules and immunologically active portions of immunoglobulin molecules, i.e., molecules that contain an antigen binding site that specifically binds (immunoreacts with) an antigen, such as a polypeptide complex. Such antibodies include, e.g. polyclonal, monoclonal, chimeric, single chain, Fab and F(ab′)2 fragments, and an Fab expression library. In specific embodiments, antibodies to human ortholog complexes.

Various procedures known within the art may be used for the production of polyclonal or monoclonal antibodies. For example, for the production of polyclonal antibodies, various suitable host animals (e.g., rabbit, goat, mouse or other mammal) may be immunized by injection with the native protein, or a synthetic variant thereof, or a derivative of the foregoing. An appropriate immunogenic preparation can contain, for example, recombinantly expressed polypeptide complex. Alternatively, the immunogenic polypeptides or complex may be chemically synthesized, as discussed above. The preparation can further include an adjuvant. Various adjuvants used to increase the immunological response include, e.g., Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory agents. If desired, the antibody molecules directed against complex can be isolated from the mammal (e.g., from the blood) and further purified by well known techniques, such as protein A chromatography to obtain the IgG fraction.

The term “monoclonal antibody” or “monoclonal antibody composition”, as used herein, refers to a population of antibody molecules that contain only one species of an antigen binding site capable of immunoreacting with a particular epitope of a polypeptide complex. A monoclonal antibody composition thus typically displays a single binding affinity for a particular protein with which it immunoreacts. For preparation of monoclonal antibodies directed towards a particular complex, or polypeptide, any technique that provides for the production of antibody molecules by continuous cell line culture may be utilized. Such techniques include, e.g., the hybridoma technique (see Kohler & Milstein, Nature 256: 495-497 (1975)); the trioma technique; the human B-cell hybridoma technique (see Kozbor, et al., Immunol Today 4: 72 (1983)); and the EBV hybridoma technique to produce human monoclonal antibodies (see Cole, et al., In: Monoclonal Antibodies and Cancer Therapy, Alan R. Liss, Inc., (1985) pp. 77-96). If desired, human monoclonal antibodies may be prepared by using human hybridomas (see Cote, et al., Proc. Natl. Acad. Sci. USA 80: 2026-2030 (1983)) or by transforming human B-cells with Epstein Barr Virus in vitro (see Cole, et al., In: Monoclonal Antibodies and Cancer Therapy, supra).

Methods can be adapted for the construction of F_{ab} expression libraries (see e.g., Huse, et al., Science 246: 1275-1281 (1989)) to allow rapid and effective identification of monoclonal F_{ab} fragments with the desired specificity for the desired protein or derivatives, fragments, analogs or homologs thereof. Non-human antibodies can be “humanized” by techniques well known in the art. See e.g., U.S. Pat. No. 5,225,539. Antibody fragments that contain the idiotypes to a polypeptide or polypeptide complex may be produced by techniques known in the art including, e.g.: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Chimeric and humanized monoclonal antibodies against the polypeptide complexes, or polypeptides, described herein are also within the scope of the invention, and can be produced by recombinant DNA techniques known in the art, for example using methods described in PCT International Application No. PCT/US86/02269; European Patent Application No. 184,187; European Patent Application No. 171,496; European Patent Application No. 173,494; PCT International Publication No. WO 86/01533; U.S. Pat. No. 4,816,567; European Patent Application No. 125,023; Better et al., Science 240: 1041-1043 (1988); Liu et al., Proc. Nat. Acad. Sci USA 84: 3439-3443 (1987); Liu et al., J. Immunol. 139: 3521-3526 (1987); Sun et al., Proc. Nat. Acad. Sci. USA 84: 214-218 (1987); Nishimura et al., Cancer Res. 47 999-1005 (1987); Wood et al., Nature 314: 446-449 (1985); Shaw et al., J. Natl. Cancer Inst. 80: 1553-1559 (1988); Morrison, Science 229: 1202-1207 (1985); Oi et al., BioTechniques 4: 214 (1986); U.S. Pat. No. 5,225,539; Jones et al., Nature 321: 552-525 (1986); Verhoeyan et al., Science 239: 1534 (1988); and Beidler et al., J. Immunol. 141: 4053-4060 (1988).

Methods for the screening of antibodies that possess the desired specificity include, e.g., enzyme-linked immunosorbent assay (ELISA) and other immunologically-mediated techniques known within the art. For example, selection of antibodies that are specific to a particular domain of a polypeptide complex is facilitated by generation of hybridomas that bind to the complex, or fragment thereof, possessing such a domain.

In certain embodiments of the invention, antibodies specific for the polypeptide complexes described herein may be used in various methods, such as detection of complex, and identification of agents which disrupt complexes. These methods are described in more detail, below. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Polypeptide complex-specific, or polypeptide-specific antibodies, can also be used to isolate complexes using standard techniques, such as affinity chromatography or immunoprecipitation. Thus, the antibodies disclosed herein can facilitate the purification of specific polypeptide complexes from cells, as well as recombinantly produced complexes expressed in host cells.

Kits

In a specific embodiment, the invention provides kits containing a reagent, for example, an antibody described above, which can specifically detect a polypeptide complex, or a constituent polypeptide, described herein. Such kits can contain, for example, reaction vessels, reagents for detecting complex in sample, and reagents for development of detected complex, e.g. a secondary antibody coupled to a detectable marker. The label incorporated into the anti-complex, or anti-polypeptide antibody may include, e.g., a chemiluminescent, enzymatic, fluorescent, calorimetric or radioactive moiety. Kits of the present invention may be employed in diagnostic and/or clinical screening assays.

Pharmaceutical Compositions

The invention further provides pharmaceutical compositions of purified complexes suitable for administration to a subject, most preferably, a human, in the treatment of disorders involving altered levels of such complexes. Such preparations include a therapeutically-effective amount of a complex, and a pharmaceutically acceptable carrier. As utilized herein, the term “pharmaceutically acceptable” means approved by a regulatory agency of the Federal or a state government or listed in the U.S. Pharmacopoeia or other generally recognized pharmacopoeia for use in animals and, more particularly, in humans. The term “carrier” refers to a diluent, adjuvant, excipient, or vehicle with which the therapeutic is administered and includes, but is not limited to such sterile liquids as water and oils.

The therapeutic amount of a complex which will be effective in the treatment of a particular disorder or condition will depend on the nature of the disorder or condition, and may be determined by standard clinical techniques by those of average skill within the art. In addition, in vitro assays may optionally be employed to help identify optimal dosage ranges. The precise dose to be employed in the formulation will also depend on the route of administration, and the overall seriousness of the disease or disorder, and should be decided according to the judgment of the practitioner and each patient's circumstances. However, suitable dosage ranges for intravenous administration of the complexes of the present invention are generally about 20-500 micrograms (μg) of active compound per kilogram (Kg) body weight. Suitable dosage ranges for intranasal administration are generally about 0.01 pg/kg body weight to 1 mg/kg body weight. Effective doses may be extrapolated from dose-response curves derived from in vitro or animal model test systems. Suppositories generally contain active ingredient in the range of 0.5% to 10% by weight; oral formulations preferably contain 10% to 95% active ingredient.

Various delivery systems are known and can be used to administer a pharmaceutical preparation of a complex of the invention including, e.g.: (i) encapsulation in liposomes, microparticles, microcapsules; (ii) recombinant cells capable of expressing the polypeptides of the complex; (iii) receptor-mediated endocytosis (see, e.g., Wu et al., J. Biol. Chem. 262: 4429-4432 (1987)); (iv) construction of a nucleic acid encoding the polypeptides of the complex as part of a retroviral or other vector, and the like.

Methods of administration include, e.g., intradermal, intramuscular, intraperitoneal, intravenous, subcutaneous, intranasal, epidural, and oral routes. The pharmaceutical preparations of the present invention may be administered by any convenient route, for example by infusion or bolus injection, by absorption through epithelial or mucocutaneous linings (e.g., oral mucosa, rectal and intestinal mucosa, etc.) and may be administered together with other biologically-active agents. Administration can be systemic or local. In addition, it may be advantageous to administer the pharmaceutical preparation into the central nervous system by any suitable route, including intraventricular and intrathecal injection. Intraventricular injection may be facilitated by an intraventricular catheter attached to a reservoir (e.g., an Ommaya reservoir). Pulmonary administration may also be employed by use of an inhaler or nebulizer, and formulation with an aerosolizing agent. It may also be desirable to administer the pharmaceutical preparation locally to the area in need of treatment; this may be achieved by, for example, and not by way of limitation, local infusion during surgery, topical application, by injection, by means of a catheter, by means of a suppository, or by means of an implant. In a specific embodiment, administration may be by direct injection at the site (or former site) of a malignant tumor or neoplastic or pre-neoplastic tissue.

Alternatively, pharmaceutical preparations of the invention may be delivered in a vesicle, in particular a liposome, (see, e.g., Langer, Science 249:1527-1533 (1990)) or via a controlled release system including, e.g., a delivery pump (see, e.g., Saudek, et al., New Engl. J. Med. 321: 574 (1989) and a semi-permeable polymeric material (see, e.g., Howard, et al., J. Neurosurg. 71: 105 (1989)). Additionally, the controlled release system can be placed in proximity of the therapeutic target (e.g., the brain), thus requiring only a fraction of the systemic dose. See, e.g., Goodson, In: Medical Applications of Controlled Release, 1984 (CRC Press, Bocca Raton, Fla.).

Screening, Diagnostic, and Therapeutic Methods

The invention further provides methods of identifying an agent which modulate formation or stability a polypeptide complex described herein. By modulate is meant to increase or decrease the rate at which the complex is assembled or dissembled, or to increase or decrease the stability of an assembled complex. Thus, an agent can be tested for its ability to disrupt a complex, or to promote formation or stability of a complex.

In one embodiment, the invention provides a method of identifying an agent that promotes disruption of a complex. The method includes providing a polypeptide complex, contacting the complex with a test agent, and detecting the presence of a polypeptide displaced from the complex. The presence of displaced polypeptide indicates the disruption of the complex by the agent. In some embodiments, the complex is a human ortholog complex, as described above, which includes “bait” and “prey” proteins selected from those recited in Table 1. In other embodiments, the complex contains at least one vesicle trafficking associated protein, as described above, and is selected from the complexes recited in Tables 2 and 3. In other embodiments, the complex contains at least one phosphatase I protein, as described above, and is the complex recited in Tables 4 and 6. In yet another embodiment, the complex contains at least one calcium binding protein, as described above, and is selected from the complexes recited in Tables 7 and 8. Agents that disrupt complexes of the invention may present novel modulators of cell processes and pathways in which the complexes participate. For example, agents which disrupt complexes involving microtubule proteins may be selected as potential anti-fungal therapeutics.

Any compound or other molecule (or mixture or aggregate thereof) can be used as a test agent. In some embodiments, the agent can be a small peptide, or other small molecule produced by e.g., combinatorial synthetic methods known in the art. Disruption of the complex by the test agent, e.g. binding of the agent to the complex, can be determined using art recognized methods, e.g., detection of polypeptide using polypeptide-specific antibodies, as described above. Bound agents can alternatively be identified by comparing the relative electrophoretic mobility of complexes exposed to the test agent to the mobility of complexes that have not been exposed to the test agent.

Agents identified in the screening assays can be further tested for their ability to alter and/or modulate cellular functions, particularly those functions in which the complex has been implicated. These functions include, e.g., control of vesicle trafficking, phosphatase I activity, and calcium binding, etc., as described in detail above.

In another embodiment, the invention provides methods for inhibiting the interaction of a polypeptide with a ligand, by contacting a complex of the protein and the ligand with an agent that disrupts the complex, as described above. In certain embodiments, the polypeptides are vesicle trafficking-associated proteins, phosphatase I proteins, or calcium binding proteins. In certain embodiments, the ligand is an interacting polypeptide, and the polypeptide and ligands are selected from those recited in Table 1. Inhibition of complex formation allows for modulation of cellular functions and pathways in which the targeted complexes participate.

In another embodiment, the invention provides a method for identifying a polypeptide complex in a subject. The method includes the steps of providing a biological sample from the subject, detecting, if present, the level of polypeptide complex. In some embodiments, the complex includes a first polypeptide (a “bait” polypeptide) selected from the polypeptides recited in Table 1, column 2, and a second polypeptide (“prey” polypeptide) selected from the polypeptides recited in Table 1, column 3. Any suitable biological sample potentially containing the complex may be employed, e.g. blood, urine, cerebral-spinal fluid, plasma, etc. Complexes may be detected by, e.g., using complex-specific antibodies as described above. The method provides for diagnostic screening, including in the clinical setting, using, e.g., the kits described above.

In still another embodiment, the present invention provides methods for detecting a polypeptide in a biological sample, by providing a biological sample containing the polypeptide, contacting the sample with a corresponding polypeptide to form a complex under suitable conditions, and detecting the presence of the complex. A complex will form if the sample does, indeed, contain the first polypeptide. In some embodiments, the polypeptide being detecting is a “prey” protein selected from the polypeptides recited in Table 1, column 3, and is detected by complexing with the corresponding “bait” protein recited in Table 1, column 2. Conversely, in other embodiments the polypeptide being detected is the “bait” protein. Alternatively, a yeast “bait” or “prey” ortholog may be employed to form a chimeric complex with the polypeptide in the biological sample.

In still another embodiment, the invention provides methods for removing a first polypeptide from a biological sample by contacting the biological sample with the corresponding second peptide to form a complex under conditions suitable for such formation. The complex is then removed from the sample, effectively removing the first polypeptide. As with the methods of detecting polypeptide described above, the polypeptide being removed may be either a “bait” or “prey” protein, and the second corresponding polypeptide used to remove it may be either a yeast or human ortholog polypeptide.

Methods of determining altered expression of a polypeptide in a subject, e.g. for diagnostic purposes, are also provided by the invention. Altered expression of proteins involved in cell processes and pathways can lead to deleterious effects in the subject. Altered expression of a polypeptide in a given pathway leads to altered formation of complexes which include the polypeptide, hence providing a means for indirect detection of the polypeptide level. The method involves providing a biological sample from a subject, measuring the level of a polypeptide complex of the invention in the sample, and comparing the level to the level of complex in a reference sample having known polypeptide expression. A higher or lower complex level in the sample versus the reference indicates altered expression of either of the polypeptides that forms the complex. The detection of altered expression of a polypeptide can be use to diagnose a given disease state, and or used to identify a subject with a predisposition for a disease state. Any suitable reference sample may be employed, but preferably the test sample and the reference sample are derived from the same medium, e.g. both are urine, etc. The reference sample should be suitably representative of the level polypeptide expressed in a control population.

In a certain embodiment, the polypeptide complex contains a “bait” polypeptide selected from the polypeptides recited in Table 1, column 2, and a “prey” polypeptide selected from the polypeptides recited in Table 1, column 3.

The invention further provides methods for treating or preventing a disease or disorder involving altered levels of a polypeptide complex, or polypeptide, disclosed herein, by administering to a subject a therapeutically-effective amount of at least one molecule that modulates the function of the complex. As discussed above, altered levels of polypeptide complexes described herein may be implicated in disease states resulting from a deviation in normal function of the pathway in which a complex is implicated. For example, altered levels of the observed complex between PPP1CC and PPP1CC-NOV1 may be implicated in disruptions in phosphatase activity, for example. In subjects with a deleteriously high level of complex, modulation may consist, for example, by administering an agent which disrupts the complex, or an agent which does not disrupt, but down-regulates, the functional activity of the complex. Alternatively, modulation in subjects with a deleteriously low level of complex may be achieved by pharmaceutical administration of complex, constituent polypeptide, or an agent which up-regulates the functional activity of complex. Pharmaceutical preparations suitable for administration of complex are described above.

In one embodiment, a disease or disorder involving altered levels of a polypeptide selected from the polypeptides recited in Table 1, column 2 or the corresponding polypeptides in column 3, is treated by administering a molecule that modulates the function of the polypeptide. In certain embodiments, the modulating molecule is the corresponding polypeptide, e.g. administering a “prey” protein corresponding to a “bait” protein modulates the latter by forming a complex with it.

The details of one or more embodiments of the invention are set forth in the description above. Although any methods and materials similar or equivalent to those described herein can be used in the practice of the present invention, the preferred methods and materials are now described. For example, additional interactions can be identified using other two-hybrid systems (i e. using a LexA binding domain fusion or HIS3 as a reporter gene), including variables such as different protein domains or genomic activation domain libraries. Other features, objects, and advantages of the invention will be apparent from the description and from the claims.

We claim:

1. A purified complex comprising a first polypeptide and a second polypeptide, wherein
said first polypeptide comprises an amino acid sequence of a polypeptide selected from the group consisting of the polypeptides recited in Table 1, column 2, and wherein said second polypeptide comprises an amino acid sequence of the corresponding polypeptide recited in Table 1, column 3; or
said first and second polypeptide comprise the amino acid sequences of a first polypeptide-second polypeptide complex selected from the group consisting of NAPA1-IP2, NAPA1-SYN16, NAPA1-SNAP29, NAPA1-SYN4, NAPA1-LZIP, NAPA1-SNAP25A, PPP1CC-NOV1, PPP1CC-NOV2, PPP1CC-NOV3, PPP1CC-NOV4, PPP1CC-NOV5, PPP1CC-PPP1R5, PPP1CC-KIAA0305, PPP1CC-STAU, PPP1CC-53BP2, PPP1CC-PPP1R10, S100A1-NOV6, S100A1-NOV7, S100A1-NOV8, S100A1-fibrinogen, S100A1-RanBPM, S100A1-profilin II-SV, S100B-NOV9, S100B-NOV10, S100B-NOV11, S100B-fibrinogen, S100B-KIAA0629, S100B-ATP6N1, S100B-synphilin I, S100B-NQO2, S100B-FHOS, S100B-S100A9, and S100B-S100A6.

2. The complex of claim 1, wherein said first polypeptide is selected from the group consisting of the polypeptides recited in Table 1, column 2, and wherein said second polypeptide is the corresponding polypeptide recited in Table 1, column 3.

3. The complex of claim 1, wherein said first polypeptide is labeled.

4. The complex of claim 1, wherein said second polypeptide is labeled.

5. The complex of claim 3, wherein said second polypeptide is labeled.

6. The complex of claim 1, wherein said first polypeptide is selected from the group of polypeptides recited in Table 1, column 2, which are denoted in Protein Pair ID: 1-11, and wherein said second polypeptide is the corresponding polypeptide selected from the group consisting of the polypeptides recited in Table 1, column 3, which are denoted as Protein Pair 1-11.

7. The complex of claim 1, wherein said first polypeptide is selected from the group of polypeptides recited in Table 1, column 2, which are denoted in Protein Pair ID: 12-58, and wherein said second polypeptide is the corresponding polypeptide selected from the group consisting of the polypeptides recited in Table 1, column 3, which are denoted in Protein Pair ID: 12-58.

8. The complex of claim 1, wherein said first polypeptide is selected from the group of polypeptides recited in Table 1, column 2, which are denoted in Protein Pair ID: 12-24, and wherein said second polypeptide is the corresponding polypeptide selected from the group consisting of the polypeptides recited in Table 1, column 3, which are denoted in Protein Pair ID: 12-24.

9. The complex of claim 1, wherein said first polypeptide is selected from the group of polypeptides recited in Table 1, column 2, which are denoted in Protein Pair ID: 25-42, and wherein said second polypeptide is the corresponding polypeptide selected from the group consisting of the polypeptides recited in Table 1, column 3, which are denoted in Protein Pair ID: 25-42.

10. The complex of claim 1, wherein said first polypeptide is selected from the group of polypeptides recited in Table 1, column 2, which are denoted in Protein Pair ID: 43-58, and wherein said second polypeptide is the corresponding polypeptide selected from the group consisting of the polypeptides recited in Table 1, column 3, which are denoted in Protein Pair ID: 43-58.

11. The complex of claim 1, wherein said first and second polypeptide comprise the amino acid sequences of a first polypeptide-second polypeptide selected from the group consisting of NAPA 1-IP2, NAPA 1-SYN16, NAPA 1-SNAP29, NAPA 1-SYN4, NAPA 1-LZIP, NAPA 1-SNAP25A, PPP1CC-NOV1, PPP1CC-NOV2, PPP1CC-NOV3, PPP1CC-NOV4, PPP1CC-NOV5, PPP1CC-PPP1R5, PPP1CC-KIAA0305, PPP1CC-STAU, PPP1CC-53BP2, PPP1CC-PPP1R10, S100A1-NOV6, S100A1-NOV7, S100A1-NOV8, S100A1-fibrinogen, S100A1-RanBPM, S100A1-profilin II-SV, S100B-NOV9, S100B-NOV10, S100B-NOV11, S100B-fibrinogen, S100B-KIAA0629, S100B-ATP6N1, S100B-synphilin I, S100B-NQO2, S100B-FHOS, S100B-S100A9, and S100B-S100A6.

12. A purified complex comprising a first polypeptide and a second polypeptide, wherein said first polypeptide comprises a region of amino acids of a polypeptide selected from the group consisting of the polypeptides recited in Table 1, column 2 sufficient to allow said first polypeptide to bind said second polypeptide, and wherein said second polypeptide comprises a region of amino acids of the corresponding polypeptide recited in Table 1, column 3 sufficient to bind said first polypeptide.

13. A chimeric polypeptide comprising six or more amino acids of the first polypeptide of claim 1 covalently linked to six or more amino acids of the second polypeptide of claim 1.

14. A nucleic acid encoding the chimeric polypeptide of claim 13.

15. A vector comprising the nucleic acid of claim 14.

16. A cell comprising the vector of claim 15.

17. An antibody which specifically binds the complex of claim 1.

18. The antibody of claim 17, wherein said antibody binds to the complex of claim 1 with higher affinity than it binds to said first or second polypeptide when said polypeptides are not complexed.

19. A pharmaceutical composition comprising the complex of claim 1.

20. A kit comprising in one or more containers a reagent which can specifically detect the complex of claim 1.

21. The kit of claim 20, wherein said reagent is selected from the group consisting of an antibody specific for said complex, an antibody specific for said first polypeptide, and an antibody specific for said second polypeptide.

22. A method of identifying an agent which disrupts a polypeptide complex, the method comprising:
(a) providing the complex of claim 1;
(b) contacting the complex with a test agent; and
(c) detecting the presence of a polypeptide displaced from said complex,
wherein the presence of displaced polypeptide indicates said agent disrupts said complex.

23. A method for identifying an agent which disrupts a polypeptide complex comprising at least one vesicle trafficking-associated protein, phosphatase I protein, or calcium binding protein, the method comprising:
(a) providing the complex of claim 11;
(b) contacting said complex with a test agent; and
(c) detecting the presence of a polypeptide displaced from said complex,
wherein the presence of displaced polypeptide indicates said agent disrupts said complex.

24. A method for inhibiting interaction of a vesicle trafficking-associated protein, with a ligand, the method comprising:
contacting a complex comprising said protein and said ligand with an agent that disrupts said complex, wherein said complex is selected from the group consisting of NAPA1-IP2, NAPA1-SYN16, NAPA1-SNAP29, NAPA1-SYN4, NAPA1-LZIP, and NAPA1-SNAP25A,
thereby inhibiting interaction of said protein with said ligand.

25. A method for inhibiting interaction of a phosphatase I protein with a ligand, the the method comprising:
contacting a complex comprising said protein and said ligand with an agent that disrupts said complex, wherein said complex is selected from the group consisting of PPP1CC-NOV1, PPP1CC-NOV2, PPP1CC-NOV3, PPP1CC-NOV4, PPP1CC-NOV5, PPP1CC-PPP1R5, PPP1CC-KIAA0305, PPP1CC-STAU, PPP1CC-53BP2, and PPP1CC-PPP1R10,
thereby inhibiting interaction of said protein with said ligand.

26. A method for inhibiting interaction of a calcium binding protein with a ligand, said method comprising the step of:
contacting a complex comprising said protein and said ligand with an agent that disrupts said complex, wherein said complex is selected from the group consisting of S100A1-NOV6, S100A1-NOV7, S100A1-NOV8, S100A1-fibrinogen, S100A1-RanBPM, S100A1-profilin 11-SV, S100B-NOV9, S100B-NOV10, S100B-NOV11, S100B-fibrinogen, S100B-KIAA0629, S100B-ATP6N1, S100B-synphilin I, S100B-NQO2, S100B-FHOS, S100B-S100A9, and S100B-S100A6,
thereby inhibiting interaction of said protein with said ligand.

27. A method of identifying a polypeptide complex in a subject, the method comprising:
(a) providing a biological sample from said subject; and
(b) detecting, if present, the polypeptide complex of claim 1 in said sample,
thereby identifying said complex.

28. A method of detecting a polypeptide in a biological sample, the method comprising:
(a) providing a biological sample comprising the first polypeptide of claim 1;
(b) contacting said biological sample with the second polypeptide of claim 1 under conditions suitable for formation of a complex comprising said first and second polypeptides; and
(c) detecting the presence of the complex of said first and second polypeptide,
wherein the presence of said complex indicates the presence of said first polypeptide in said sample.

29. A method of detecting a polypeptide in a biological sample, the method comprising:
(a) providing a biological sample comprising the second polypeptide of claim 1;
(b) contacting said biological sample with the first polypeptide of claim 1 under conditions suitable for formation of a complex comprising said first and second polypeptides; and
(c) detecting the presence of the complex of said first and second polypeptide,
wherein the presence of said complex indicates the presence of said second polypeptide in said sample.

30. A method of removing a polypeptide from a biological sample, the method comprising:
(a) providing a biological sample comprising the first polypeptide of claim 1;
(b) contacting said biological sample with the second polypeptide of claim 1 under conditions suitable for formation of a complex comprising said first and second polypeptide; and
(c) removing said complex from said sample,
thereby removing said first polypeptide from said sample.

31. A method of determining altered expression of a polypeptide in a subject, the method comprising:
(a) providing a biological sample from said subject,
(b) measuring the level of the complex of claim 1 in said sample; and
(c) comparing the level of said complex from step (b) to the level of said complex in a reference sample whose level of the complex of claim 1 is known,
thereby determining whether said subject has altered expression of said first or second polypeptide.

32. A method of treating or preventing a disease or disorder involving altered levels of the complex of claim 1, the method comprising:
administering a therapeutically-effective amount of least one molecule that modulates the function of said complex to a subject in need thereof.

33. An isolated polypeptide comprising an amino acid sequence selected from the group consisting of:
(a) a mature form of an amino acid sequence encoded by the nucleic acid sequence selected from the group consisting of SEQ ID NOS: 1, 2, 3, 4, 5, 6, 7, 8, 9, 11, 12, and 13;
(b) a variant of a mature form of an amino acid sequence encoded by the nucleic acid sequence selected from the group consisting of SEQ ID NOS:1, 2, 3, 4, 5, 6, 7, 8, 9, 11, 12, and 13, wherein one or more amino acid residues in said variant differs from the amino acid sequence of said mature form, provided that said variant differs in no more than 15% of the amino acid residues from the amino acid sequence of said mature form;
(c) an amino acid sequence encoded by the nucleic acid sequence selected from the group consisting of SEQ ID NOS:1, 2, 3, 4, 5, 6, 7, 8, 9, 11, 12, and 13; and
(d) a variant of an amino acid sequence encoded by the nucleic acid sequence selected from the group consisting of SEQ ID NOS: 1, 2, 3, 4, 5, 6, 7, 8, 9, 11, 12, and 13 wherein one or more amino acid residues in said variant differs from the amino acid sequence of said mature form, provided that said variant differs in no more than 15% of amino acid residues from said amino acid sequence.

34 The polypeptide of claim 33, wherein said polypeptide comprises the amino acid sequence of a naturally-occurring allelic variant of an amino acid sequence encoded by the nucleic acid sequence selected from the group consisting of SEQ ID NOS:1, 2, 3, 4, 5, 6, 7, 8, 9, 11, 12, and 13.

35. The polypeptide of claim 34, wherein said allelic variant comprises an amino acid sequence that is the translation of a nucleic acid sequence differing by a single nucleotide from a nucleic acid sequence selected from the group consisting of SEQ ID NOS: 1, 2, 3, 4, 5, 6, 7, 8, 9, 11, 12, and 13.

36. The polypeptide of claim 33, wherein the amino acid sequence of said variant comprises a conservative amino acid substitution.

37. An isolated nucleic acid molecule comprising a nucleotide sequence selected from the group consisting of
(a) a nucleotide sequence selected from the group consisting of SEQ ID NOS:1, 2, 3, 4, 5, 6, 7, 8, 9, 11, 12, and 13;
(b) a nucleotide sequence differing by one or more nucleotides from a nucleotide sequence selected from the group consisting of SEQ ID NOS: 1, 2, 3, 4, 5, 6, 7, 8, 9, 11, 12, and 13, provided that no more than 20% of the nucleotides differ from said nucleotide sequence;
(c) a nucleic acid fragment of (a); and
(d) a nucleic acid fragment of (b).

38. The nucleic acid molecule of claim 37, wherein said nucleic acid molecule hybridizes under stringent conditions to a nucleotide sequence chosen from the group consisting of SEQ ID NOS: 1, 2, 3, 4, 5, 6, 7, 8, 9, 11, 12, and 13, or a complement of said nucleotide sequence.

39. A vector comprising the nucleic acid molecule of claim 37.

40. The vector of claim 39, further comprising a promoter operably-linked to said nucleic acid molecule.

41. A cell comprising the vector of claim 40.

42. An antibody that immunospecifically-binds to the polypeptide of claim 33.

43. The antibody of claim 42, wherein said antibody is a monoclonal antibody.

44. The antibody of claim 42, wherein the antibody is a humanized antibody.
